(19) United States
(12) Patent Application Publication
(10) Pub. No.: US 2020/0405218 A1
(43) Pub. Date: Dec. 31, 2020

(54) DEVICE, SYSTEM AND METHOD FOR DETERMINING A PHYSIOLOGICAL PARAMETER

(71) Applicant: AUT Ventures Limited, Auckland (NZ)

(72) Inventors: Miguel Jose Jo AVILA, Auckland (NZ); Eric Russell HELMS, Auckland (NZ); Parn Naruenart Wangdee JONES, Auckland (NZ); Tassanai PARITTOTOKKAPORN, Auckland (NZ)

(21) Appl. No.: 16/955,677

(22) PCT Filed: Dec. 18, 2018
(86) PCT No.: PCT/NZ2018/050179

§ 371 Date: Jun. 18, 2020

(30) Foreign Application Priority Data
Dec. 18, 2017 (NZ) 738446

(51) Int. Cl. A61B 5/00

(52) U.S. Cl. CPC A61B 5/4519; A61B 5/4509; A61B 5/4872; A61B 5/0004; A61B 2562/14; A61B 5/6832; A61B 5/0051; A61B 2562/0204; A61B 5/0022

(57) ABSTRACT

Described is a device comprising a transducer; a mounting assembly coupled to the transducer and configured to mount the transducer adjacent to or in contact with a portion of a user's body; the transducer configured to transmit an acoustic signal to a portion of the user's body and detect a reflected acoustic signal; and a processor in electronic communication with the transducer, the processor configured to determine a physiological parameter based on the reflected acoustic signal. Also described is a system using the device for determining a physiological parameter of a user.

### FIELD OF THE INVENTION

The present invention relates to a device, system and/or method for determining a physiological parameter. In particular, but not limited to, the present invention relates to a device, system and/or method for determining a muscular parameter of a person. The device and system may also be used to assist a user in exercising.

### BACKGROUND

Physiological parameters of humans are measured in various fields such as for example in the medical industry and the exercise and fitness industry. Exercising has become increasingly popular in the public due to the increased awareness of and pursuit of a healthier lifestyle. Physiological monitoring of a user is becoming more popular in order to assess fitness levels or to assess the type or quality of workout being performed.

Fitbit or JawBone are two example activity tracker devices that can be used to monitor physiological parameters of a user pre, during or post workouts, as well as during other times. Some of the most common parameters that are measured by currently available devices are heart rate, steps (pedometer), sleep patterns and calories burnt. These devices and other devices on the market generally comprise a wearable device that can synchronise with an app on a mobile device and/or remote at least one remote server to store data and process data. The physiological parameters (i.e. fitness data) can be processed in a number of different ways to provide a user with an indication of fitness level or the activities performed by a user. However, these systems are generalised indication of fitness and do not provide any specific muscular parameter measurement or information.

Physiological parameter monitoring is also used in the training of elite athletes or in high level sports training (i.e. professional sports), such as for example rugby, MMA, bodybuilding etc. High level sports training often involves carefully tracking and measuring specific physiological parameters in order to optimise workouts. Some of the exemplary parameters that are measured are heart rate, calories burnt, body fat amount, body composition, as well as some muscular parameters such as for example muscle activation. Devices used to measure muscular parameters are known and used often in high level sports training (i.e. professional sports) context. Some commonly used devices are electromyography (EMG) devices which are not portable, expensive and complex to set up and use.

Currently there is a need for a portable, easily useable and accurate system to determine a physiological parameter, in particular to determine muscular parameters. In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

For the purpose of this specification, where method steps are described in sequence, the sequence does not necessarily mean that the steps are to be chronologically ordered in that sequence, unless there is no other logical manner of interpreting the sequence.

### SUMMARY OF THE INVENTION

It is an object of the at least some embodiments of the invention to provide a device, system and/or method for determining a physiological parameter e.g. muscular parameter of a person, and/or at least provides the public with a useful choice. Other objects of the invention may become apparent from the following description which is given by way of example only.

The present invention relates generally to a device e.g. a sensor that can be used to determine one or more muscular parameters for use in an exercise system that can assist a user in performing one or more exercise routines or workouts. The present invention may also relate to an exercise system that may incorporate a sensor device for determining one or more muscular parameters and a communication device, such as for example a mobile device, to assist a user in performing one or more exercise routines.

In a first aspect the present invention broadly consists in a device comprising: a transducer, a mounting assembly coupled to the transducer and configured to mount the transducer adjacent to or in contact with a portion of a user's body, the transducer configured to transmit an acoustic signal to a portion of the user's body and detect a reflected acoustic signal, and a processor in electronic communication with the transducer, the processor configured to determine a physiological parameter based on the reflected acoustic signal.

The acoustic signal transmitted by the transducer is a low frequency acoustic signal or a vibroacoustic signal.

The acoustic signal is of a frequency less than or equal to 250 Hz.

The acoustic signal is of a frequency greater than or equal to 50 Hz and less than or equal to 500 Hz.

The acoustic signal is of a frequency greater than or equal to 70 Hz and less than or equal to 350 Hz.

The acoustic signal is of a frequency that corresponds substantially to the resonant frequency of muscle tissue. The frequency of the acoustic signals being substantially similar to the resonant frequency of the muscle tissue provides improve response and reduces noise within the signal. A greater portion of the signal is reflected when the frequency range is substantially similar to the resonant frequency of muscle tissue.

The transducer is a vibroacoustic transducer capable of generating and receiving a vibroacoustic signal.

The transducer comprises: a transmitter configured to generate and transmit an acoustic signal, and a receiver configured to receive a reflected acoustic signal.

The transmitter and the receiver are spaced apart from each other.

The transmitter and receiver are spaced apart from each other by 1 mm to 100 mm.

The transmitter and receiver are spaced apart from each other by 7 mm to 10 mm.

Preferably the transmitter and receiver are spaced apart 8 mm. Spacing the transmitter and receiver apart by 8 mm reduces noise being introduced and detected by the receiver but also improves the signal received at the receiver. The distance between the transmitter and receiver reduces vibrations from the transmitter interfering with or causing noise at the receiver.

The transducer comprises a housing, the transmitter and the receiver being located within the housing and being spaced apart from each other, wherein the housing comprises an opening positioned within the housing and adjacent the receiver.

The opening defines an air gap adjacent the receiver. The air gap acts to amplify acoustic signals received at the receiver and also reduces noise being detected by the receiver.

The device comprises a coupling layer disposed between the transducer and a portion of the user's body adjacent the transducer.

The coupling layer is formed from a resilient material. The coupling layer acts as a damper and absorbs noise. The coupling layer also improves contact between the transmitter and the skin to improve acoustic signal transmission.

The coupling layer is formed from at least one or a combination of: silicone material, elastomer, elastomeric material, rubberised material, silicone rubber material or a thermoplastic material.

The coupling layer is adhered or attached to a user proximal face of the housing.

The coupling layer is substantially tacky. The coupling layer may alternatively include a skin friendly adhesive layer.

The coupling layer can be removably attached to a portion of the user's body or a face of the housing.

The coupling comprises an aperture within it, the aperture being aligned with the opening in the housing when the coupling layer is adhered or attached to the housing.

The at least one physiological parameter is determined based on a mathematical relationship between the physiological parameter and reflected acoustic signal.

The at least one physiological parameter is a muscular parameter or a fat parameter or a bone density parameter.

The muscular parameter is any one of: muscle density, muscle fatigue, muscle activation or muscle engagement.

The device comprises a wireless communication unit, the wireless communication unit is configured to communicate the reflected acoustic signals to a processor for processing and identifying one or more physiological parameters.

The wireless communication unit is in electronic communication with the receiver, the receiver transmitting the reflected acoustic signals to the wireless communication unit for transmittal to a processor or server.

The device comprises one or more feedback units that are configured to provide either a visual feedback or audible feedback or haptic feedback to the user based on the determined muscular parameter.

The mathematical relationship is a proportional relationship between an amplitude of the reflected acoustic signal and a muscle density, wherein muscle density is proportional to the amplitude of the reflected acoustic signal.

The device is a non-invasive device for use in determining at least one physiological parameter.

The one or more indicators is one or more of an LED, a speaker or a vibrating unit. The mounting assembly is at least one of: a strap that can be wrapped about a portion or a muscle of the user's body, a sleeve incorporating the device and capable of being attached about a muscle or a portion of a user's body, a biocompatible adhesive that is capable of removable attachment of the device to a muscle or a portion of the user's body.

The strap is an elastic strap that is reduces muscle contraction constraint i.e. the strap does not constrain the muscle from contracting or changing shape. The strap is elastic to allow movement of the muscle as it contracts.

The mounting assembly is coupled to the housing of the transducer.

The mounting assembly comprises a garment, wherein the transducer is mounted on or within the garment or within a pocket disposed on the garment. The garment may be a shirt, a singlet, a t shirt or shorts or briefs. The garment can be worn by the user in use such that the transducer is brought adjacent a muscle or muscle group.

In another aspect, the present invention broadly consists in a system for determining a physiological parameter of a user comprising: the device according to any one of the earlier statements, and a remote processor in communication with the device, the processor receiving the reflected acoustic signal or a signal indicative of the reflected acoustic signal and the processor further configured to determine at least one physiological parameter based on a parameter of the reflected acoustic signal, wherein the processor is separate from the device.

The physiological parameter is a muscular parameter.

The muscular parameter is muscle density and wherein the processor is configured to determine muscle density based on an area under each peak in the reflected acoustic signal. Muscular density may be determined based on an amplitude of the reflected acoustic signal or based on an absolute value of the amplitude of the reflected acoustic signal.

The muscular parameter is muscle activation and wherein the processor is configured to determine the muscle density based on the occurrence of a peak within the reflected sound signal.

The muscular parameter is muscle fatigue and wherein the processor is configured to determine muscle fatigue based on change in amplitude of two or more peaks within a predefined time period.

A peak in the reflected acoustic signal is a signal comprising an amplitude above a threshold.

The system comprising: a mobile device arranged in wireless communication with the device according any one of the above statements, and the mobile device providing feedback to the user based on one or more muscular parameters detected from the reflected acoustic signal by the processor.

The mobile device is configured to receive the reflected acoustic signals and determine one or more physiological parameters e.g. muscular parameters based on the reflected acoustic signal. For example the mobile device may be configured to process the reflected acoustic signals to determine a muscular parameter and display the muscular parameter or one or more other parameters based on the muscular parameters to the user.

The remote processor is disposed within a mobile device.

The remote processor is disposed within a cloud server system.

The mobile device comprises a user interface for communication with the user, the mobile device presenting one or more exercise routines to the user via the user interface, the mobile device receiving a selection of one or more exercise routines to be performed by the user and the mobile device providing feedback regarding an aspect of the one or more exercise routines based on the one or more determined muscular parameters.

The aspect of the one or more exercise routines may be one or more of amount of weight to lift, number of repetitions, a change in the exercise routine duration, or stop exercising due to an injury.

The one or more indicators of the device are configured to provide feedback regarding an aspect of the one or more exercise routines, wherein the activation of the indicators is controlled based on the one or more determined muscular parameters.

The activation of the indicators is controlled by the mobile device.

A system according to any one of the earlier statements comprising a storage system, the storage system comprising a server in electronic communication with the device or the mobile device or both the mobile device and the device, the server comprising at least a processor and a memory unit, and wherein the storage system is configured to store one or more a) muscular parameters determined based on the reflected acoustic signal, b) reflected acoustic signals or c) user details.

A system according to any one of the earlier statements comprising; a server, the server is arranged in wireless communication with the mobile device or the device according to any one of the earlier statements, or both the mobile device and the device, and the server is a cloud based server.

In a further aspect, the present invention broadly consists in a sensor for use in an exercise system comprising the device according to any one of the earlier statements.

In a further aspect, the present invention broadly consists in an exercise system comprising the system according to any one of the earlier statements, wherein the exercise system is configured to assist a user with performance of an exercise routine.

In a further aspect, the present invention broadly consists in a method for determining a physiological parameter comprising the steps of: providing an acoustic signal to a muscle or muscle group, receiving a reflected acoustic signal; and processing the reflected acoustic signal to determine a physiological parameter based on the reflected acoustic signal.

The reflected acoustic signal is reflected off at least the muscle or muscle group of a user.

The physiological parameter is a muscular parameter i.e. a parameter related to a muscle or muscle group of the user.

The acoustic signal provided to the muscle or muscle group is a vibration or mechanical signal.

The acoustic signal provided to the muscle or muscle group is within the vibroacoustic frequency range.

The acoustic signal is of a frequency less than or equal to 250 Hz.

The acoustic signal is of a frequency greater than or equal to 50 Hz and less than or equal to 500 Hz.

The acoustic signal is of a frequency greater than or equal to 70 Hz and less than or equal to 350 Hz.

The acoustic signal is of a frequency that corresponds substantially to the resonant frequency of muscle tissue. The frequency of the acoustic signals being substantially similar to the resonant frequency of the muscle tissue provides improve response and reduces noise within the signal. A greater portion of the signal is reflected when the frequency range is substantially similar to the resonant frequency of muscle tissue.

The step of processing further comprises the additional steps of: receiving a reflected acoustic signal, converting the received reflected acoustic signal (i.e. an electrical signal indicative of the reflected acoustic signal) to a logarithmic scale, and determining a muscular parameter based on the logarithmic representation of the reflected acoustic signal.

The logarithmic representation is dB, wherein the reflected acoustic signal is converted to dB (i.e. decibels).

A muscular parameter is based on a parameter of the logarithmic representation of the reflected acoustic signal.

The method comprises the additional step of filtering the received reflected acoustic signal prior to converting the received reflected acoustic signal to a logarithmic scale.

The step of filtering comprises applying a root mean square (RMS) filter to the reflected acoustic signals received by the wearable device.

The method of determining a physiological parameter is performed by a controller or processor disposed on or in the wearable device.

The method of determining a physiological parameter is stored as a set of computer readable and executable instructions within a memory unit associated with the wearable device.

Alternatively the method of determining a physiological parameter may be executed by the mobile device or a server or a combination of the wearable device, mobile device and server.

The method of determining a physiological parameter comprises the additional steps of: comparing the determined physiological parameter to a threshold; and providing an indication to a user based on the relationship of the determined physiological parameter and the threshold.

The threshold is related to a physiological parameter or a muscular parameter or effort put in by the user.

The threshold is predefined by the user. The threshold may be stored in a memory unit associated with the wearable device or may be stored on a mobile device or a server.

The indication may be any one or more of a visual, audible or haptic indication. The indication acts as feedback to the user regarding performance of an exercise or workout by a user. Alternatively the indication acts as feedback to the user regarding a specific physiological parameter e.g. muscular parameter that is being detected. Preferably the indication is provided during exercise and the threshold may relate to a selected exercise routine or selected workout.

The method of determining a physiological parameter further comprises the step of transmitting the determined physiological parameter(s) to a server or a processing and storage system. The processing and storage system comprises at least a processor and a memory unit to store physiological parameters and/or other user related data. The server or the processing and storage system are remote to the wearable device.

The method determining a physiological parameter comprises the additional step of performing a calibration method or routine for a user to determine one or more baseline physiological parameters.

The baseline physiological parameter may be baseline muscular parameter. A threshold can be set based on the baseline muscular parameter and any determined muscular parameter may be compared against the threshold or the baseline muscular parameter. The baseline muscular parameter may relate to a one repetition maximum value (one rep max). A baseline muscular parameter may be determined for each muscle or muscle group.

The method of calibration comprises the steps of: initiating a calibration routine, providing an acoustic signal, receiving a reflected acoustic signal, determining a baseline muscular parameter (or physiological parameter) based on the reflected acoustic signal, and storing the baseline muscular parameter.

The step of initiating a calibration routine comprises providing a notification or message to a user to perform a one rep max of a lift or exercise, and determining a muscular parameter related to the one rep max to provide the baseline muscular parameter.

The calibration method may be executed a plurality of times to determine an average baseline muscular parameter (or baseline physiological parameter).

The method of calibration is preferably performed by the wearable device or the mobile device or a combination of the wearable device and mobile device.

In a further aspect, the present invention broadly consists in a non transient computer readable medium containing program instructions for causing a computer or processor to perform a method of determining a physiological parameter as per the method described earlier.

In a further aspect, the present invention broadly consists in a method of assisting a user to perform an exercise routine comprising the steps of: receiving an exercise routine selection; transmitting acoustic signals to a muscle or muscle group; receiving a reflected acoustic signal, processing a reflected acoustic signal to determine one or more physiological parameters; and providing feedback to the user regarding the selected exercise routine based on the determined one or more physiological parameters.

The one or more physiological parameters may be a muscular parameter related to a muscle.

The acoustic signal is a signal in the vibroacoustic frequency range.

The method of exercising comprises providing real time feedback to the user regarding the selected feedback. The feedback may be visual, audible, haptic or a combination thereof.

The method of exercising comprises the additional step of comparing the one or more determined physiological parameters with one or more thresholds, and providing a feedback based on the relationship between the one or more determined physiological parameters and the one or more thresholds. The thresholds may be user selected or may be predefined and related to an exercise routine.

The method of exercising may comprise any one or more of the steps or features of the method of determining a physiological parameter as described earlier.

The term ‘comprising’ as used in this specification and claims means ‘consisting at least in part of’. When interpreting statements in this specification and claims which include the term ‘comprising’, other features besides the features prefaced by this term in each statement can also be present. Related terms such as ‘comprise’ and ‘comprised’ are to be interpreted in a similar manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The term acoustic signal as used herein relates to a sound signal or sound wave or vibrations or a vibration signal or a mechanical wave that can be transmitted and/or received. An acoustic signal can be a sound or vibration signal that can be generated and transmitted by the transmitter.

The term reflected acoustic signal as used herein refers to a reflected sound signal or a reflected vibration signal. The term reflected acoustic signal also incorporates an electronic signal indicative of a reflected acoustic signal. In this specification where the reflected acoustic signal is processed etc. refers to processing an electrical signal indicative of the actual vibrations measured by the transducer.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The term user as used herein with respect to the wearable device described herein, means a person that wears the wearable device. Participants as used herein means any other person or persons that may communicate with an element of the systems described herein.

The terms muscle parameter and muscular parameter and variations thereof mean the same and can be interchangeably used.

The term physiological parameter as used herein means a parameter (i.e. value or variable or factor) that is measurable or mathematically determinable and related to the human body i.e. to human physiology. The physiological parameter may be directly measured or derived or determined using a mathematical process.

As used herein the term ‘(s)’ following a noun means the plural and/or singular form of that noun.

As used herein the term ‘and/or’ means ‘and’ or ‘or’, or where the context allows both.

The invention consists in the foregoing and also envisages constructions of which the following gives examples only.

As used herein “(s)” following a noun means the plural and/or singular forms of the noun.

In the following description, specific details are given to provide a thorough understanding of the embodiments. However, it will be understood by one of ordinary skill in the art that the embodiments may be practiced without these specific details. For example, software modules, functions, circuits, etc., may be shown in block diagrams in order not to obscure the embodiments in unnecessary detail. In other instances, well-known modules, structures and techniques may not be shown in detail in order not to obscure the embodiments.

Also, it is noted that the embodiments may be described as a process that is depicted as a flowchart, a flow diagram, a structure diagram, or a block diagram. Although a flowchart may describe the operations as a sequential process, many of the operations can be performed in parallel or concurrently. In addition, the order of the operations may be rearranged. A process is terminated when its operations are completed. A process may correspond to a method, a function, a procedure, a subroutine, a subprogram, etc., in a computer program. When a process corresponds to a function, its termination corresponds to a return of the function to the calling function or a main function.

Aspects of the systems and methods described below may be operable on any type of general purpose computer system or computing device, including, but not limited to, a desktop, laptop, notebook, tablet or mobile device. The term “mobile device” includes, but is not limited to, a wireless device, a mobile phone, a smart phone, a mobile communication device, a user communication device, personal digital assistant, mobile hand-held computer, a laptop computer, an electronic book reader and reading devices capable of reading electronic contents and/or other types of mobile devices typically carried by individuals and/or having some form of communication capabilities (e.g., wireless, infrared, short-range radio, etc.).

### BRIEF DESCRIPTION OF THE DRAWINGS

Exemplary embodiments of the invention will now be described by way of example only and with reference to the drawings, in which:

FIG. 1 shows an exemplary embodiment of an exercise system that can be used to assist with exercise and/or assist with determining a physiological parameter of a user;

FIG. 1a shows an exemplary embodiment a server that may be used as part of the exercise system of FIG. 1;

FIG. 2 shows a schematic diagram of the wearable device from FIG. 1;

FIGS. 3a, 3b and 3c show exploded views of the main components of the wearable device of FIG. 2;

FIG. 4a shows a bottom view i.e. from the perspective of the face that contacts the user of the wearable device of FIGS. 3a, 3b and 3c;

FIG. 4b shows an elastic strap used to mount the wearable device on a muscle, wherein the strap accommodates movement of the muscle as the muscle contracts;

FIGS. 5a and 5b show the wearable device in use being attached to a portion of the user's body;

FIG. 6 shows a flow chart for a method of processing the acoustic signals to determine physiological parameters e.g. muscular parameters;

FIG. 7a-7c show three exemplary plots of Force applied by a user during an exercise and the corresponding reflected acoustic signal that is measured;

FIGS. 8a-8b show exemplary plots that are generated during exercise, the plot showing acoustic signals in dB;

FIGS. 9, 10a and 10b show exemplary plots of different types of workouts during multiple repetitions of an exercise e.g. bicep curls;

FIG. 11 shows an exemplary method of assisting a user during exercise;

FIG. 12 shows an example method of using an exercise system including a wearable device, during exercising;

FIG. 13 shows an exemplary embodiment of a wearable device that can be used as part of a system, such as the system shown in FIG. 1; and

FIGS. 14a to 14h show an exploded view, bottom view, front view, top view, left side view, right side view, bottom isometric view and top isometric view of an exemplary wearable device.

### DETAILED DESCRIPTION

The disclosure relates to a device, system and/or method for determining a muscular parameter of a person. The determined muscular parameters can be used to assist a user in performing one or more exercise routines. The device uses an acoustic signal to determine a muscular parameter of a user. The muscular parameters can also be used by other persons or users to assist

The disclosure also relates to a sensor device that can be used to determine a muscular parameter of a user and be used in an exercise system. The sensor device uses acoustic signals to determine a muscular parameter. The disclosure also relates to an exercise system that comprises at least a device for determining a physiological parameter e.g. a muscular parameter and a processor, wherein the exercise system assists a user in performing one or more exercise routines based on the determined physiological parameter.

The disclosure also relates to a method of determining a physiological parameter e.g. a muscular parameter and a method of for assisting a user to perform one or more exercise routines.

In its most general form the exercise system comprises a device and a processor in electronic communication. The device is preferably a wearable device and the processor is preferably a remote processor that is separated from the device. The device may comprise its own onboard processor that is configured for signal processing. The device is configured to transmit an acoustic signal to a portion of a user's body, receive a reflected acoustic signal from a portion of the user's body and transmit the reflected acoustic signal (or an electronic signal indicative of the reflected acoustic signal) to the processor. The processor is configured to determine a muscular parameter based on the reflected acoustic signal (or based on the electronic signal indicative of the reflected acoustic signal).

### System Details

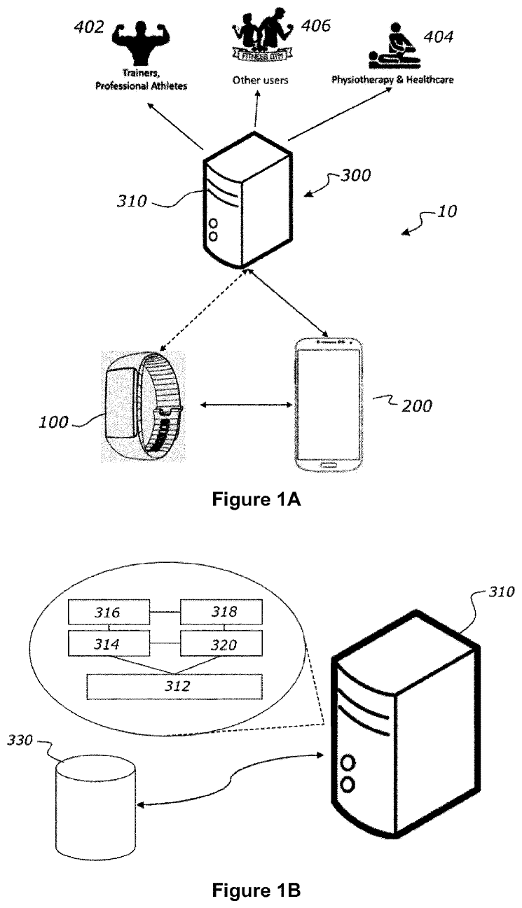

Referring to FIG. 1, an exemplary embodiment of system 10 is illustrated. The system 10 can be used as an exercise system 10 to assist with exercising and/or to assist a user in assessing any exercise performed by the user. The exercise system 10 is configured to assist a user in performing one or more exercise routines based on a determined physiological parameter. The exercise system 10 further comprises a system generally for determining a physiological parameter such as for example a muscular parameter of user.

The exercise system 10 comprises a wearable device 100 that is configured to be positioned in contact with a portion of the user's body. The wearable device 100 illustrated in FIG. 1 is a wearable device that can be worn on a portion of a user's body e.g. a limb. The device 100 is configured to generate and transmit an acoustic signal to a portion of the user's body and receive a reflected acoustic from a portion of the user's body. The device 100 is preferably a wearable device that is worn on a portion of a user's body e.g. a limb of a user. The wearable device 100 is placed in contact with a muscle or muscle group of a user. The wearable device 100 comprises an on-board processor that is configured to determine a physiological parameter e.g. a muscle related parameter based on the reflected acoustic signal.

In the illustrated embodiment the exercise system 10 further comprises a mobile device 200 and a processing and storage system 300. The system also includes a plurality of other participants 402-406 i.e. users that can utilise information such as for example the physiological parameters of the user. FIG. 1 shows exemplary system participants e.g. trainers 402, physiotherapists 404 or other users 406 who may also have their own wearable devices. In some instances the storage system 300 can also the user using the device 100 to communicate various parameters e.g. muscular parameters or performance of a workout with their specific friends or on various social networks. The system 300 and the wearable device 100 may include modules (hardware and/or software) that allow social network connectivity. In some configurations the user may use mobile device 200 to communicate with the various participants or social networks to transmit muscular parameters or other physiological parameters. In a further alternative the wearable device 100 may comprise appropriate electronics to allow direct communication with electronic devices or mobile devices e.g. smartphone or tablets associated with the other participants to transmit physiological parameters e.g. muscular parameters.

In use, a single wearable device 100 may be a general purpose device that can be used by any user by wearing the device 100. Alternatively the wearable device 100 may be customised and associated with a single user.

The other participants 402-406 can at least access physiological parameters of one or more users that may use a wearable device 100. Access to physiological parameters e.g. muscular parameters may be limited for privacy reasons and may only be accessed by an appropriate secure protocol e.g. a password protocol. In one example a trainer 402 may be able to remotely access muscular parameters of a particular client (i.e. user of the wearable device) in order to assist the user with exercising. The various participants may also be able to connect with each other, and thus the system 10 also creates a social network for users of the system and allows information exchange e.g. improved exercise routines, sharing of exercise results or sharing of muscular parameter information. Participants can access the physiological parameters from the system 300 where various user related information is stored.

The mobile device 200 is in electronic communication with the device 100. Preferably the mobile device 200 can wirelessly communicate with the device 100 to receive an electronic signal indicative of the physiological parameters or receive the muscular parameters from the device 100. In the illustrated embodiment the wearable device 100 determines the physiological parameter. Alternatively the mobile device 200 may be configured to receive electronic signals indicative of the reflected acoustic signals, and process these to determine a physiological parameter.

The mobile device 200, as per this embodiment, may also be used as a user interface to present information to a user e.g. physiological parameters or provide an indication of an exercise being performed or provide feedback regarding performance of an exercise by the user.

The mobile device 200 may be any suitable mobile device such as for example a smartphone or tablet or a laptop. The mobile device 200 comprises a processing unit, a memory unit and a communications unit. The mobile device 200 as shown in FIG. 1, is a smartphone such as for example an iPhone.

In the illustrated embodiment the processing and storage system 300 preferably comprises a server 310. The server 310 comprises at least a processor and an associated memory unit or a plurality of memory banks. The memory unit may be internal to the server or may be external or remote. The memory unit may be cloud based provided by a cloud service provider or may be a suitable hardware memory unit e.g. a hard drive or magnetic tape or solid state memory or any other suitable memory unit. The server 310 may be a remote server for example a cloud server provided by a cloud service provider. Alternatively the server 310 may be implemented as part of a server farm or may be a local server for example implemented as a desktop computer or a laptop computer or any other suitable computing device. The processing and storage system 300 in the illustrated example is a cloud server system that also includes a storage unit for example a memory unit or a plurality of memory banks such as hard drives or tape drives or any other suitable type of storage unit.

The server 310 is configured to store user related information such as for example user credentials or a user profile. In this embodiment, the server 310 is also configured to store physiological parameters of a user. The physiological parameters are determined based on the reflected acoustic signals and may be determined by the device 100 in this embodiment. The physiological parameters may alternatively be determined by the mobile device 200. In the illustrated embodiment the wearable device 100 sends the physiological parameters or information indicative thereof to the mobile device 200, which in turn transmits this information to the server 310 for storage. Alternatively the wearable device 100 may be in direct electronic communication with the server 310.

The stored information on the server 310 can be sent to the mobile device 200 for presenting to the user. For example the server 310 may store physiological parameters for any suitable time frame e.g. months or years etc. In one example the measured physiological parameters e.g. muscle density can be presented on the mobile device 200.

FIG. 1a shows a schematic diagram of an exemplary server 310 that can be part of or form the processing and storage system 300. The mobile device 200 is preferably arranged in a client server relationship with the server 310. The other participant devices 402-406 e.g. computing devices or mobile devices are arranged in a client server relationship with the server 310. In at least one configuration the wearable device 100 may also be arranged in a client server relationship with the server 310, such that the wearable device 100 can directly transmit and/or receive information from the server 310.

The server 310 comprises suitable components necessary to receive, store and execute appropriate computer instructions. The components may include a processing unit 312, read-only memory (ROM) 314, random access memory (RAM) 316, and input/output devices such as disk drives 318, input devices such as an Ethernet port, a USB port, etc. and communications links 320. The server 310 includes instructions that may be included in ROM 314, RAM 316 or disk drives 318 and may be executed by the processing unit 312. There may be provided a plurality of communication links 320 which may variously connect to one or more computing devices such as a server, personal computers, terminals, wireless or handheld computing devices, such as for example the other participants of the system 402, 404, 406. At least one of a plurality of communications link may be connected to an external computing network through a telephone line or other type of communications link.

The server 310 may include storage devices such as a disk drive 318 which may encompass solid state drives, hard disk drives, optical drives or magnetic tape drives. The server 310 may use a single disk drive or multiple disk drives. The server 310 may also have a suitable operating system 322 which resides on the disk drive or in the ROM of the server 310.

The system 300 may comprise a database 330 residing on a disk or other storage device which is arranged to store at least one record providing a link between a user and the user's muscular parameter (or other physiological parameter). The database 330 may be a relational database or any other suitable database structure. The database 330 may also store other information such as for example user credentials, user IDs, exercise routines that can be selected to be performed, calibration data for each user and other information. The database may also categorize information based on the user, such that information associated with each user is related within the database for quick access.

The server 310 may comprise software that defines computer readable instructions that define the server 310 operations. For example the server 310 comprises software e.g. a software application that can be executed by the processor 312 to receive and store various information such as for example physiological parameters of the user or exercise performance or exercises/workouts done by the user etc. The server 310 may also comprise software stored in a memory unit that can be executed by the processor 312 to allow the server 310 to present results graphically e.g. create graphs or plots to illustrate various parameters associated with exercise such as for example exercise performance. The software may also allow other participants 402-406 to extract physiological parameters of the user and/or access information related to a user, or a user's performance of exercises and/or other activities.

The wearable device 100 is configured to wirelessly communicate with the mobile device 200 using any suitable wireless communication protocol. The wearable device 100 uses short distance wireless communication protocols such as for example low energy Bluetooth or an infrared protocol or any other suitable local wireless communication protocol. The mobile device 200 is configured to wirelessly communicate with the processing and storage system 300 (i.e. at least the server 310), in particular at least with the processor of the processing and storage system 300 (i.e. server 310). The mobile device 200 is arranged in a range for two-way communication between elements of the processing and storage system 300 such that information can be sent and received from the processing and storage system 300. Example communication protocols between the mobile device 200 and server 310 can be 3G or 4G or WiFi or WAN or any other suitable communication protocol.

The wearable device 100 is configured to generate an acoustic signal and receive a reflected acoustic signal from a portion of the user's body. The wearable device 100 is further configured to process the reflected acoustic signals or at least an electronic signal indicative of the reflected acoustic signal to determine a physiological parameter e.g. a muscular parameter of a user. The wearable device 100 comprises at least an onboard processor in communication with a transducer such that physiological parameters of a user can be determined. The physiological parameters are transmitted to the mobile device 200. The mobile device 200 is configured to at least display the various physiological parameters. In one example each measurement/determination of the physiological parameters is presented to the user on the mobile device 200 e.g. on a user interface of the mobile device 200. The physiological parameters may be determined and presented to the user quickly. In one example at least the determination of the physiological parameters is performed in substantially real time i.e. in real time or close to real time. One example of substantially real time is less than every 5-8 seconds.

The mobile device 200 in turn transmits the physiological parameters to the processing and storage system 300 for storage and access at a later point. The processing and storage system 300 is also configured to aggregate various physiological parameter values and other information related to the user e.g. exercises performed, how well an exercise was performed etc.

The mobile device 200 may perform some initial pre-processing such as filtering the raw signal received from the wearable device 100. The mobile device 200 may further amplify the reflected acoustic signal received from the wearable device 100 and may also perform some additional smoothing functions to improve the quality of the reflected acoustic signal received from the wearable device 100. For example the mobile device 200 may improve the signal to noise ratio of the reflected acoustic signal.

In an alternative embodiment the mobile device 200 may be configured to receive the reflected acoustic signals (or an electronic signal representative of the reflected acoustic signals) and process these signals to determine a physiological parameter. The mobile device 200 may be configured to present the determined physiological parameters to the user. The physiological parameters may be determined and presented in real time or substantially real time e.g. within a time period of less than 5-8 seconds.

In a further embodiment the server 310 may be configured to determine the physiological parameters from the reflected acoustic signal. The server 310 may receive the reflected acoustic signals (or from an electronic signal indicative of the reflected acoustic signal) from the wearable device 100 or via the mobile device. The server 310 may also comprise software (e.g. a software application or software program) that is executable by the processor, of the server 310, and is configured to process electronic signals representative of the reflected acoustic signals (i.e. reflected acoustic signals) received from the mobile device 200 to identify one or more physiological parameters e.g. one or more muscular parameters. The determined muscular parameters can be transmitted back to the mobile device 200 for communication to the user and/or can be stored in a memory unit of the server 310.

### Wearable Device

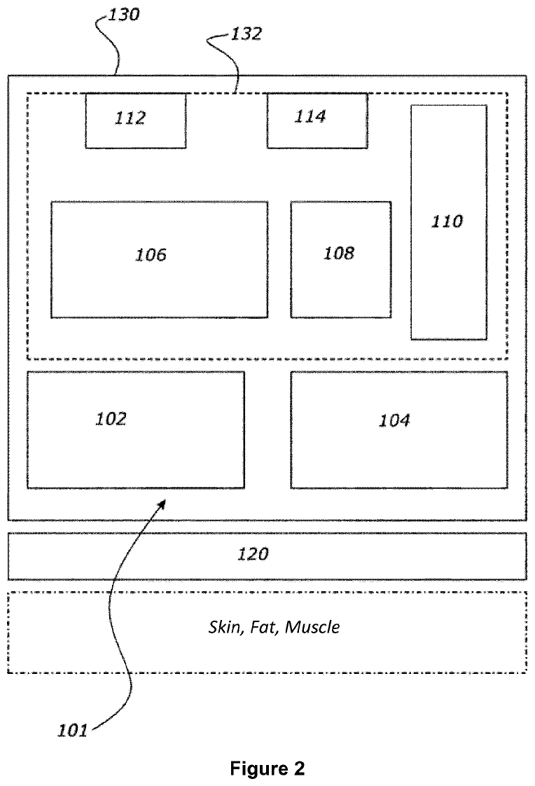

FIG. 2 shows a schematic diagram of a device for determining a muscular parameter. More specifically FIG. 2 shows an embodiment of a wearable device 100 that can be used to determine a muscular parameter. The wearable device 100 shown in FIG. 2, an embodiment of the wearable device that can be used as part of the exercise system 10. The device 100 can be worn on a portion of the user's body for example on an arm or a leg or the chest. Preferably the device is worn to be in contact with a portion of the user's body, in particular a major muscle group. The device 100 is configured to determine one or more muscular parameters associated with one or more muscles or muscle groups of the user. Example, muscle groups may be biceps, quadriceps, triceps, pectoral muscles or any other muscles that the user would like to determine muscular parameters related to. The illustrated examples in the specification will be discussed with reference to the bicep muscle.

The wearable device 100 comprises a transducer 101 and a mounting assembly 150 coupled to the transducer 101 and configured to mount the transducer adjacent to or in contact with a portion of a user's body. The transducer is configured to transmit an acoustic signal to a portion of the user's body and detect a reflected acoustic signal.

As shown in the embodiment of FIG. 2, the wearable device 100 comprises a transmitter 102 and a receiver 104 that are positioned adjacent each other. The transducer 101 comprises the transmitter 102 and receiver 104. The transmitter 102 and the receiver 104 are spaced apart from each other in order to reduce interference and noise due to contact or clattering or vibrations being caused in the receiver 104 from the transmitter 102. As shown in FIG. 2, the wearable device 100 comprises a controller 106, a battery 108 and a communication unit 110. The wearable device 100 also comprises one or more indicators that can be used to provide feedback to the user. In the illustrated embodiment of FIG. 2, the wearable device 100 comprises a pair of indicators 112, 114.

The controller 106 is arranged in electronic communication with the transmitter 102 and is configured to control the operation of transmitter 102. The controller 106 can also control the operation the indicators 112, 114 by transmitting an appropriate control signal to one or more both indicators 112, 114 to either activate or deactivate the indicators 112, 114. The controller 106 may also control the power supplied to one or more of the components of the wearable device 100 based appropriately programmed power needs or requirements.

The transmitter 102 is preferably a vibroacoustic transmitter transmitting unit. The transmitter 102 is configured to transmit acoustic signals in any suitable acoustic signal frequency range. Preferably the transmitter is configured to transmit acoustic in the vibroacoustic frequency range of the acoustic spectrum. The transmitter is positioned to be in contact with a portion of the user's body i.e. in contact with the skin and generate an acoustic signal by vibrating a portion of the user's body the transmitter is in contact with. The transmitter 102 preferably comprises a vibroacoustic unit such as a coin vibrator or a linear vibrator or a linear piston or any other suitable vibration inducing (i.e. vibration creating) unit. The transmitter 102 creates an acoustic signal by vibrating a portion of the user's body. The transmitter 102 is preferably arranged in contact with a portion of the user's body, e.g. a muscle or muscle group of the user. Alternatively the transmitter may include a layer of material in between the contact transmitter and the skin of the user.

The transmitter 102 further comprises a suitable driving unit such as for example a motor that is mechanically coupled to disk shaped driver e.g. a coin driver. The motor can be any suitable motor e.g. a linear motor. The transmitter 102 also comprises a pulse width modulation unit (PWM unit) as part of the driving unit. The pulse width modulation unit comprises appropriate electronic circuitry and electronic components. The PWM unit is preferably in electronic communication with a controller 106 and receives actuation signals from the controller 106. The controller 106 is programmed to actuate the PWM unit, and thus activate the transmitter 102 to create an acoustic signal. A transmitter 102 is preferably arranged in direct contact with a portion of the user's body in order to maximise the transmission of vibrations to the portion of the user's body. The transmitter surface area may be anywhere between 100 mm and 200 mm. Preferably the area of contact between the skin and the transmitter 102 is approximately 125 mm-135 mm

The vibrating unit of the transmitter 102 transmits an acoustic signal in the vibroacoustic frequency range of the audio/acoustic spectrum. Vibroacoustic frequency range is defined as an acoustic signal between 50 hertz and 500 hertz more preferably the frequency range is less than 250 hertz but greater than or equal to 50 hertz even more preferably the frequency ranges between 70 hertz and 350 hertz. The vibroacoustic signal is preferable compared to other acoustic frequencies such as ultrasound because the vibroacoustic signals are inaudible to the human, penetrate deep into muscle tissue without causing any damage to the muscle tissue and are easy to generate with a low power input requirement.

Vibroacoustic waves are transmitter by the transmitter. These waves are acoustic waves that comprise vibrations i.e. mechanical waves generated by the transmitter. The transmitter uses preferably the vibroacoustic frequency as it closely aligns with the natural resonant frequency of muscle tissue. This frequency range provides an optimal response i.e. optimal level of reflected acoustic signals that can be detected by the receiver 104. The selected frequency maximises penetration of the acoustic signals and improves response. Generally acoustic signals outside the range of 50 Hz-500 Hz can either damage muscle or cause muscular breakdown or are difficult to detect or are not reflected as clearly as the selected frequency range.

The receiver 104 comprises an acoustic receiver such as for example a microphone. The microphone (i.e. receiver 104) can be any suitable microphone such as a piezoelectric microphone or electret microphone or condenser microphone or MEMS microphone or any other suitable microphone. The receiver 104 is configured detect i.e. receive a reflected acoustic signal from a portion of the user's body. The reflected acoustic signal is the reflected component of the transmitted acoustic signal that reflects off the user's body, in particular off the muscles of the user. The selected transmitter 102 and receiver 104 are substantially small to be accommodated within a housing and mounted on a user's limb. Further the selected transmitter 102 and receiver 104 are substantially low power devices.

The receiver 104 also comprises suitable interfacing circuitry that couples to the controller 106. The interfacing circuitry may also connect to the battery 108 to draw power from the battery to power the receiver 104. The interfacing circuitry comprises electronic components that are configured to receive the reflected acoustic signals and at least convert the reflected acoustic signal to an electronic signal that is indicative i.e. representative of the reflected acoustic signal from a portion of the user's body. The interfacing circuitry of the receiver 104 may also do some initial pre-processing such as some initial filtering and/or amplification of the electronic signal representative of the reflected acoustic signal. The receiver 104 is configured to transmit the electronic signal representative of the reflected acoustic signal (now referred to as the reflected acoustic signal) to the communication unit 110 for transmission to the processor 312 or mobile device 200

The controller 106 can be any suitable controller such as a microprocessor or a microcontroller or an FPGA device or an ASIC. The controller 106 may comprises electronic components for processing the electronic signals representative of the reflected acoustic signal (i.e. the reflected acoustic signal) from the receiver 104. The controller 106 comprises a processor and a memory unit. The processor can be any suitable processor e.g. a microprocessor, ASIC, FPGA or any other suitable processor. The memory unit is in electronic communication with the processor e.g. via a bus or a PCB track. The memory unit can be any suitable type of read and write memory e.g. solid state memory or flash memory. The processor and memory unit are preferably incorporated on a PCB or chip or ASIC. The controller 106 may be configured to filter the signals, smooth the signals and amplify the signal prior to transmission to the mobile device 200.

The controller 106 preferably processes the reflected acoustic signals i.e. an electronic signal indicative of the reflected acoustic signal that is received from the receiver 104. The controller is configured to determine at least one physiological parameter from the processing, for example the controller 106 is configured to determine muscular parameters. In one exemplary embodiment the controller 106 is configured to at least determine muscular density based on the reflected acoustic signal, in particular the amplitude of the reflected acoustic signal. The amplitude of the reflected acoustic signal is proportional to the voltage of the electric signal indicative of the reflected acoustic signal.

The communication unit 110 may be any suitable wireless communication unit. The communication unit 110 is configured to receive the reflected acoustic signals (i.e. an electronic signal representative or indicative of the reflected acoustic signal) from the receiver 104. The communication unit 110 in one example is a low energy Bluetooth unit that is configured to communication the reflected acoustic signals to the mobile device using Bluetooth. Alternatively the communication unit 110 may be an infrared unit or any other suitable wireless communication unit.

As shown in FIG. 2, the wearable device comprises a pair of indicators 112, 114 configured to provide feedback to the user. The feedback may be one or more of a visual, audible or haptic feedback. In the illustrated example shown in FIG. 2, the indicators 112, 114 are light emitting diodes (LEDs) that are controlled by the controller 106 to visually provide feedback to the user. The feedback will be described later. Alternatively the indicators may comprise a speaker or a buzzer or a bell for audible feedback or a vibrating actuator to provide a haptic feedback.

The wearable device 100 comprises a housing 130 that holds the components of the wearable device 100. The housing 130 may be a substantially rectangular shaped housing. Alternatively the housing 130 can be any other suitable shape e.g. substantially elliptical or cylindrical etc. The housing 130 may be formed from a rigid, corrosion resistant and robust material such as for example a plastics material or a metal. The housing 130 is also formed from a material that is suitable for use with humans and suitable for extended skin contact. In some configurations the housing 130 may be formed from a biocompatible material e.g. titanium or a biocompatible polymer material such as for example polypropylene or polyethylene. The housing 130 contains the transmitter 102, receiver 104, communication unit 110, battery 108 and the controller 106. The indicators 112, 114 may be disposed on or embedded within a portion of the housing 130. In the illustrated configuration of FIG. 2, the indicators 112, 114 are disposed on an upper side of the housing 130 that is exposed to the user. The housing 130 may be a sealed housing or alternatively may be openable to allow removal of at least the transmitter 102 and receiver 104. The housing may include an openable lid to allow access to at least the transmitter 102 and the receiver 104. The housing may be made from a suitable light, robust and biocompatible material i.e. a material that reduces any skin irritation since the wearable device 100 will be in contact with the user's skin. Some example materials of the housing are nylon, polypropylene or aluminium or stainless steel.

The wearable device 100 may optionally also comprise an electronics enclosure. In the illustrated configuration, shown in FIG. 2, the wearable device 100 comprises a separate electronics enclosure 132 that is nested within the housing. The electronics are preferably isolated from the transmitter 102 and the receiver 104 in order to prevent damage or noise being introduced into the signal.

The wearable device 100 comprises a coupling layer 120. The coupling layer 120 is disposed between the transducer 101 and a portion of the user's body adjacent the transducer 101. More specifically the coupling layer 120 is disposed on the housing 130 and is located between the housing and the skin of the user. The coupling layer 120 is preferably formed from a resilient material. The coupling layer 120 may be formed from at least one of silicone material, elastomer, elastomeric material, rubberised material, silicone rubber material or a thermoplastic material. Alternatively the coupling layer 120 may be formed from a combination of these materials. In one example the coupling layer 120 is formed from a Silicone Rubber material. One example material is a rubber with a shore hardness of approximately 60. The coupling layer 120 functions as an interface between the transducer and the skin. The coupling layer functions to improve mechanical coupling between the transmitter and the skin of the user, as well as the coupling of the receiver and the skin of the receiver. The coupling layer 120 functions to provide a sound coupling medium. The coupling layer provides a more secure contact between the skin and the wearable device thereby reducing ambient noise being measured by the receiver 104. The coupling layer helps to reduce noise being transferred to the receiver by dissipating the noise.

The coupling layer provides secure contact between the transmitter 102 and the skin of the user. The transmitter 102 may extend through the coupling layer 120 and directly contact the skin, or alternatively may contact the skin through the coupling layer. The coupling layer reduces noise transmitted by the transmitter and noise received at the receiver e.g. when the muscle contracts etc. Preferably there is an opening in the coupling layer 120 aligned with the receiver. The opening helps to amplify the acoustic signals received at the receiver as the hole can function like a funnel and guide acoustic signals toward the receiver 104.

The coupling layer 120 may be removably attached to the housing, to allow replacement of the coupling layer 120. This allows the coupling layer 120 to be changed for each new user that wears the wearable device, improving hygiene. The removable nature of the coupling layer 120 allows replacement of the coupling layer if the coupling layer 120 becomes deteriorated. Alternatively the coupling layer 120 may be permanently attached to the housing 130. The coupling layer 120 may also include a tacky surface proximal to the user to allow for improved attachment between the coupling layer and the user's skin and can help to reduce the formation of bubbles or wrinkles that may affect the transmission of acoustic signals or filtering of ambient noise.

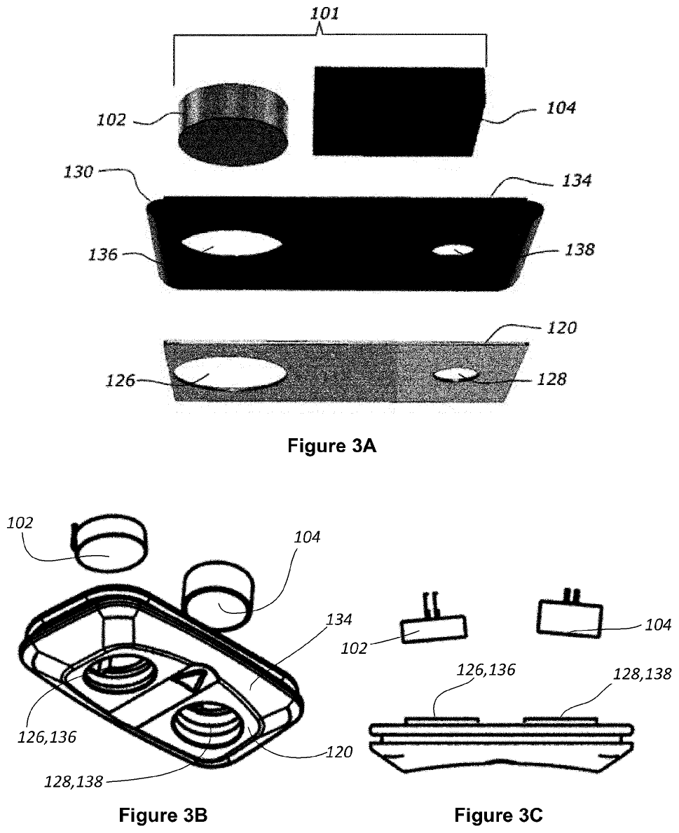

Referring to FIGS. 3a, 3b and 3c, there is a partially exploded view of the wearable device 100. FIGS. 3a, 3b and 3c show a user proximal side of the housing 134. The user proximal side 134 i.e. bottom face of the housing is arranged closest to the user in use. The coupling layer 120 is disposed on the user proximal side 134 of the housing. The user proximal side 134 comprises a first transmission opening 136 and a first receiver opening 138 within it.

The coupling layer 120 also comprises a second transmission opening 126 and a second receiver opening 128. The first transmission opening 136 and the second transmission opening 126, and the first receiver opening 138 and the second receiver opening 128 are aligned with each other when the coupling layer 120 is coupled to the housing 130. The transmitter 102 is exposed through the transmission openings 126, 136. The receiver 104 is exposed through the receiver openings 128, 138. The transmitter 102 preferably is in direct contact with the user's skin to directly transmit acoustic signals to the user's muscles. A portion of the receiver is exposed, e.g. the microphone or microphones are exposed through the receiver openings 128, 138 to allow acoustic signals reflected from a portion of the user's body e.g. a muscle to be received. The first receiver opening 138 defines an acoustic air gap that provides a channel for reflected acoustic signals to be directed into the microphone of the receiver 104. The acoustic air gap helps to reduce noise and channel acoustic signals toward the receiver thereby acting as amplify the acoustic signals. The first receiver opening 138 may have a diameter of between 2 mm and 10 mm and a depth of between 0.3 mm and 1 mm. In one example the first receiver opening 138 has a diameter of approximately 4 mm and a depth of 0.8 mm.

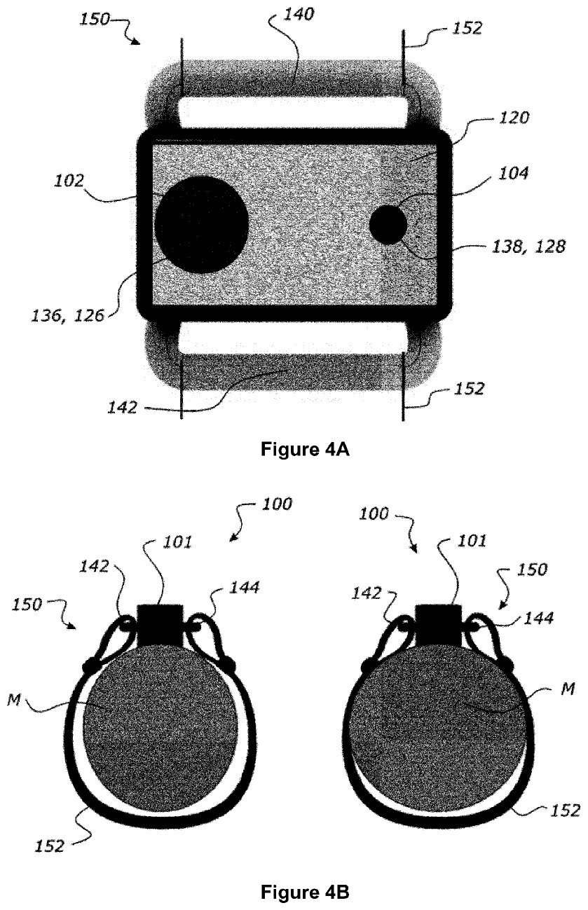

FIG. 4a shows a bottom view of the wearable device 100 i.e. a view looking onto the user proximal side. As seen in FIG. 4a, the transmitter 102 and receiver 104 are exposed. A larger area of the transmitter 102 is exposed than the receiver 104. This allows adequate skin contact between the transmitter 102 and the skin to ensure the acoustic signal, specifically a vibroacoustic signal is transmitted into the user's muscles. The transmitter 102 is spaced apart from the receiver 104, as shown in FIG. 4a. The transmitter 102 may be between 1 mm and 100 mm away from the receiver. Preferably the transmitter 102 is spaced 8 mm away from the receiver. The spacing may be measured from the two closest edges of the transmitter and receiver or may be centre to centre of the second transmission opening 126 and the second receiving opening 138. The transmitter 102 being spaced from the receiver 104 reduces noise from the transmitter 102 being detected by the receiver 104.

The wearable device 100 further comprises a mounting assembly 150 to allow a user to wear the device 100. The mounting assembly is at least one of: a strap that can be wrapped about a portion or a muscle of the user's body, a sleeve incorporating the device and capable of being attached about a muscle or a portion of a user's body, a biocompatible adhesive that is capable of removable attachment of the device to a muscle or a portion of the user's body. In the example shown in FIG. 1 the mounting assembly 150 is a strap 152. The strap 152 may be adjustable in length to allow fitting about any portion of the user's body. The strap 152 also comprises a securing structure e.g. a clip or hook and loop fasteners or any other suitable securing structure to securely attach the strap about a portion of a user's body. The strap 152 is preferably made from a soft fabric for comfort. However the strap 152 may be formed from a plastics material. In an alternative configuration the strap 152 may be formed from the same material as the housing 130 and may be integrally moulded or integrally formed with the housing 130. In the example shown in FIG. 4a, the wearable device 100 comprises a pair of projections 140, 142. The strap 152 is preferably looped through and attached to the projections 140, 142. The projections 140, 142 preferably define eyelets for the strap 152 to pass through. The projections 140, 142 form rigid loops through which the strap 152 is threaded. The strap 152 may be threaded in a manner to allow length adjustability of the strap 152. Alternatively the strap 152 may comprise a buckle through which the strap is threaded to allow adjustment of the strap length. Adjustment of the strap length allows tightening of the strap 152 to fit different sized people.

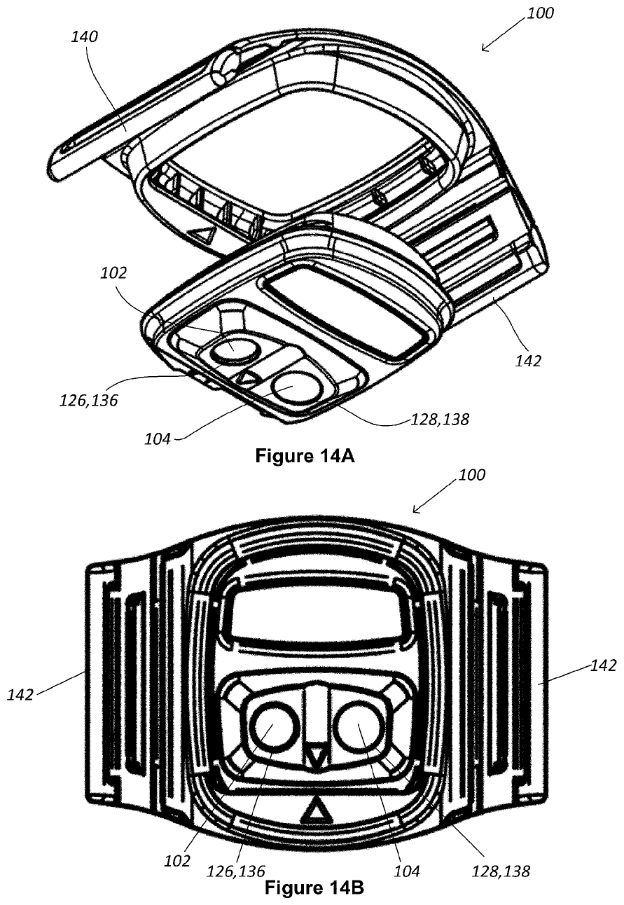

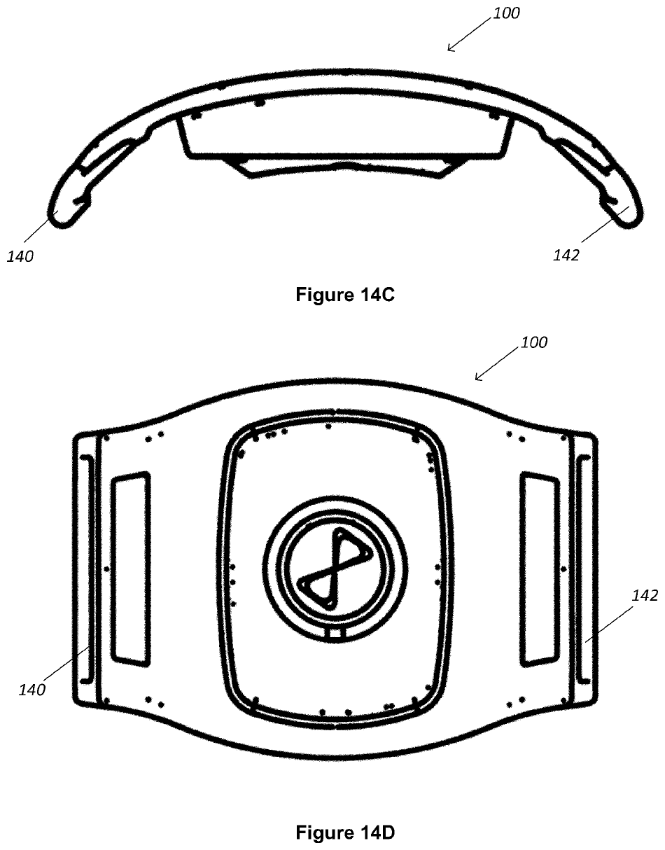

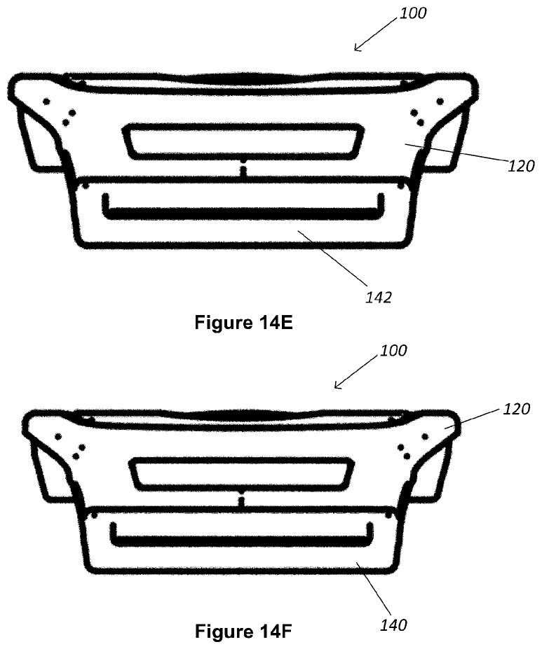

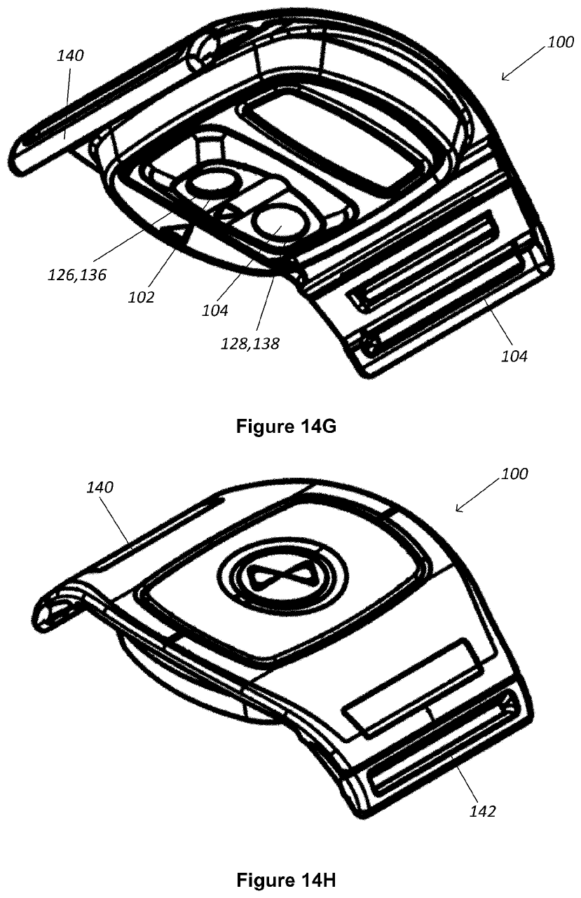

An exemplary design of the wearable device 100 is shown in FIGS. 14a to 14h.

FIG. 4b shows an exemplary configuration of the strap 152 that may be used to mount the wearable device 100 to a muscle M of the user. The muscle M can be any muscle e.g. a bicep, tricep etc. The strap 152 is preferably an elastic strap that is attached to the housing. The elastic strap 152 is formed of a resilient material. The strap 152 is elastic to accommodate muscle movement e.g. during contraction of the muscle where the muscle will effectively change shape and can increase in size. The non elastic strap causes the muscle to separate as it changes shape, whereas the elastic strap accommodates for movement or change of muscle shape. If the muscle is pushed apart, the muscular parameters are not accurately read and can lead to false positives or other erroneous readings. This compensation for muscular movement, by the elastic strap 152, allows the wearable device 100 to be maintained in contact with the muscle throughout the contraction i.e. movement. The elastic strap allows the wearable device to maintain contact and measure acoustic signals that are indicative of a muscular parameter as a user performs exercises.

Referring to FIG. 4b the figure on the left illustrates a relaxed muscle. The wearable device is positioned in contact with the muscle M. There is some room between the strap 152 and muscle. However it should be understood there may be no gap and the strap 152 may contact the muscle, but the strap is in a relaxed i.e. unstretched state. The figure to the right shows a contracted muscle that has increased in size. The strap stretches to accommodate the change in the muscle shape without compromising or restricting the muscle movement.

### Operation of the Wearable Device

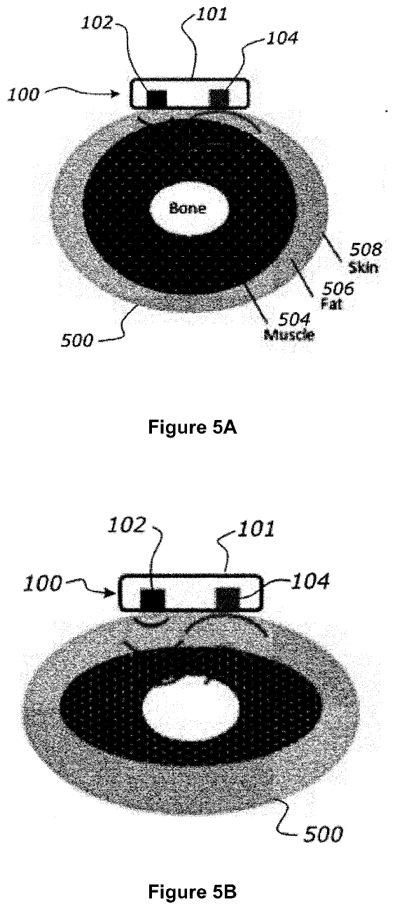

FIGS. 5a and 5b show the wearable device 100 in use being attached to a portion of the user's body. FIGS. 5a and 5b illustrate operation of the wearable device 100. FIG. 5a shows a cross section of a user's limb 500 e.g. an arm that includes bone 502, muscle 504, fat 506 and skin 508. The skin 508 is the outer most layer of the limb 500. FIG. 5a shows the limb in a relaxed position i.e. the user is relaxed. FIG. 5b shows the limb 5b in a contracted position. More specifically FIG. 5b shows the muscle 504 in a contracted position. The wearable device 100 is configured to use acoustic signals for use in determining a muscular parameter of the user. The muscular parameter can be any one or more of: muscle density, muscle fatigue, muscle activation or muscle engagement. Preferably at least muscle density is measured. Muscle density is determined based on the reflected acoustic signal (i.e. or at least based on the electrical signal indicative of the reflected acoustic signal).

As shown in FIG. 5a, the transmitter 102 transmits an acoustic signal, which in this example is a vibroacoustic signal. The signal reflects off the muscle and bone. The reflected acoustic signal is detected by the receiver 104. The intensity i.e. amplitude of the reflected acoustic signal increases as the muscle is contracted, as compared to when the muscle is relaxed. The intensity i.e. amplitude of the reflected acoustic signal, detected by the receiver is greater when the muscle is contracted as in FIG. 5b compared to when the muscle is relaxed as in FIG. 5a. When the muscle is contracted the muscle fibres bind and overlap each other, thus increasing the thickness of the muscle. This allows for greater amount of acoustic signal to reflect because in a contracted state there are less gaps between the fibres i.e. no H hand in the muscle. The contracted muscle is a more solid structure as compared to a relaxed muscle, hence greater amount of acoustic signal is reflected when the muscle is contracted. When the H hand is exposed i.e. in a relaxed state a greater amount of the acoustic signal passes through the H hand of the muscle and less signal is reflected back. The H band in a muscle is a naturally occurring gap in the muscle. The size of the gap i.e. H hand is larger when the muscle is in a contract position.

The wearable device 100 is configured to determine the physiological parameter e.g. a muscular parameter based on the reflected acoustic signal. The wearable device 100 is configured to process the reflected acoustic signals at the controller 106 and determine the physiological parameter. The determined physiological parameter can be used provided to and presented on the mobile device 200. The physiological parameters may be transmitted to and stored on the server 310. The determined physiological parameter or parameters can be used to determine other parameters e.g. performance of an exercise, the quality of the performance by the user etc. This determination of other parameters may be performed on the wearable device 100 or the mobile device 200 or the server 310 or a combination thereof. Preferably the determination of the physiological parameter is performed on the wearable device 100 or mobile device 200. The server 310 is configured to aggregate various physiological parameter measurements (i.e. values) over any specified time period. The server 310 can process the stored information to allow various types of reporting to the user via the mobile device 200.

### Processing of Acoustic Signals

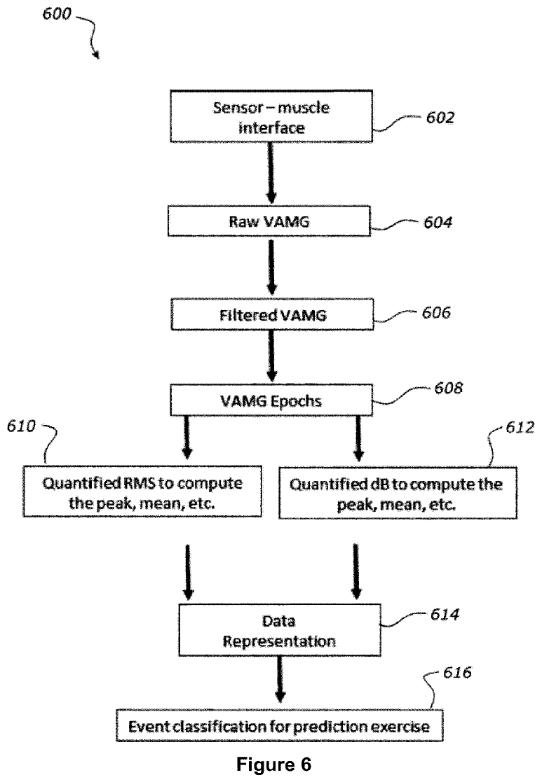

FIG. 6 shows a flow chart for a method of processing the acoustic signals 600 to determine physiological parameters e.g. muscular parameters. The method 600 as described is preferably performed either by the wearable device 100 or by a combination of the wearable device 100 and the mobile device 200. The transducer 101 (i.e. transmitter 102+receiver 104) produce a vibroacouticmyograph using the vibroacoustic signals to determine muscular parameters. FIG. 6 shows a method of processing the vibroacouticmyography. The method 600 begins at 602 that comprises the sensor-muscle interface. At step 602 the transmitter transmits the vibroacoustic signals to the muscle and the reflected vibroacoustic signals are received. Step 604 comprises receiving the raw VAMG (i.e. vibroacouticmyography) signals. Step 604 comprises the receiver 104 generating an electrical signal indicative of or representative of the reflected acoustic signal. VAMG is the recording of acoustic signals produced during muscle contractions. These become louder as the contraction force increases due to increased contraction.

Step 606 comprises filtering the reflected acoustic signal (i.e. the electrical signal representative of the reflected acoustic signal). Step 606 may comprise applying a band pass filter to allow particular bands of signals through. In one example the band for the band pass filter may be between 50 Hz and 500 Hz. Step 606 may be executed by the controller 106 or by circuitry disposed within the receiver 104.

Step 608 comprises continuously sampling the reflected acoustic signal as the user performs actions e.g. exercises such as bicep curls. Epochs refers to a change in muscular state or configuration e.g. contraction or relaxation. Step 608 comprises determining a change in state. In some instances the sampling of reflected acoustic signals may be performed over a predefined time window.

A signal sampling rate of 1 to 3 KHz can be used. Any other suitable sampling time may be used. Step 610 comprises determining a quantified RMS (root mean square) value of the electronic signal representative of the reflected acoustic signal (i.e. the reflected acoustic signal). The RMS envelope of the reflected acoustic signal may be calculated using a moving window, with each window of data calculated according to:

$$RMS = {( \frac{1}{S} {\sum }_{1}^{S} {f}^{2} ( S ) )}^{\frac{1}{2}}$$

where S equals the window length and f(s) equals data within the window.

The RMS calculation provides a measure of the intensity i.e. amplitude of the reflected acoustic signal. The RMS calculation also produces an easy to analyse and process output.

Alternative to the RMS calculation decibel processing can be applied to the reflected acoustic signals (i.e. electronic signal representative of the reflected acoustic signals) from the receiver 104. Step 612 comprises generating a quantified dB (decibel) output of the reflected acoustic signal for further processing. Step 612 comprises converting the signals from the receiver 104 into a dB value using the following definition:

$${L}_{p} = 20 {log}_{10} ( \frac{p r m s}{p r e f} ) dB$$

wherein prms is the root mean square of the measured sound pressure in pascals and pref is the standard reference sound pressure of 20 micropascals in air and 1 micropascal in water.

Step 614 comprises generating a data representation of the reflected acoustic signal using either the RMS representation as generated at step 610 or the dB representation as generated at step 612. In one form the controller 106 may be configured to generate data in both the RMS and dB representations.

The data is transmitted to a processor for event classification i.e. determination of a muscular parameter and for providing an output regarding an exercise to a user. The processor may be in the controller 106 of the wearable device or on the mobile device 200. In some configurations the server 310 may process the data to classify or identify various events. The data from the reflected acoustic signals is used to determine a muscular parameter and then used to determine performance of an exercise routine. The data regarding the muscular parameter is used to provide a feedback to the user regarding their exercise. The method 600 is preferably used during a user performing exercise and muscular parameter information is used to guide the exercise or exercise routine performed by the user. The wearable device 100 and mobile device 200 act as user feedback devices and can provide feedback to the user regarding the actions e.g. exercises, the user is performing.

The method 600 may be implemented by controller 106 of the wearable device or the mobile device 200. Alternatively at least steps 602-604 may be performed by the controller 106, step 606 may be performed by the mobile device and steps 610-616 may be performed by the server 310, since the server 310 will have greater processing power than the mobile device 200 or controller 106.

The reflected acoustic signal information can be processed to determine a muscular parameter. The muscular parameter may be any one or more of: muscle density, muscle fatigue or muscle activation. The wearable device 100 and/or mobile device may provide an indication of a good repetition or bad repetition and may also display limits of the user's muscle i.e. prior to any injury. The muscular parameter is determined based on properties of the reflected acoustic signal. For example muscular density is related to the amplitude of the reflected acoustic signal. As the amplitude increases the density increases.

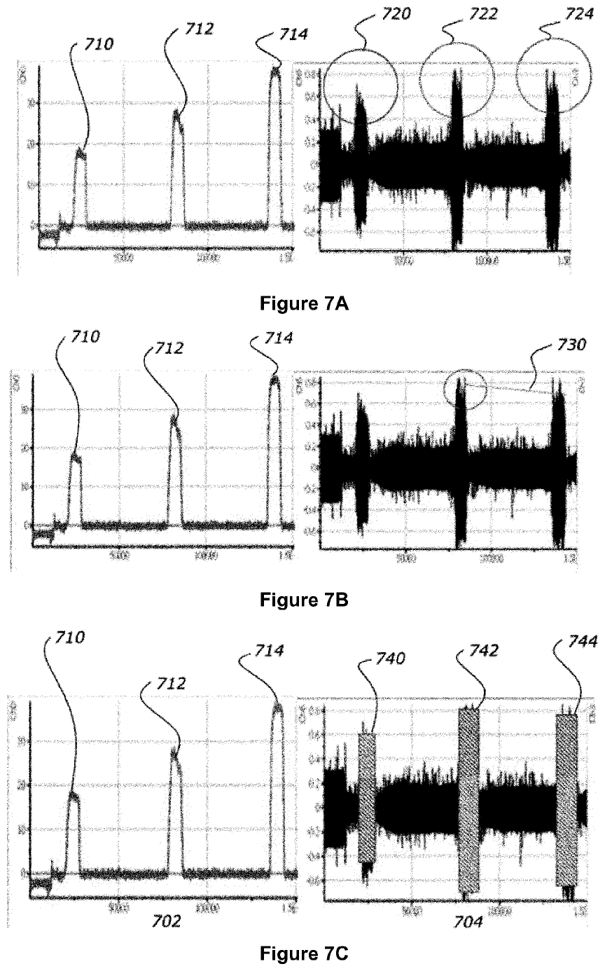

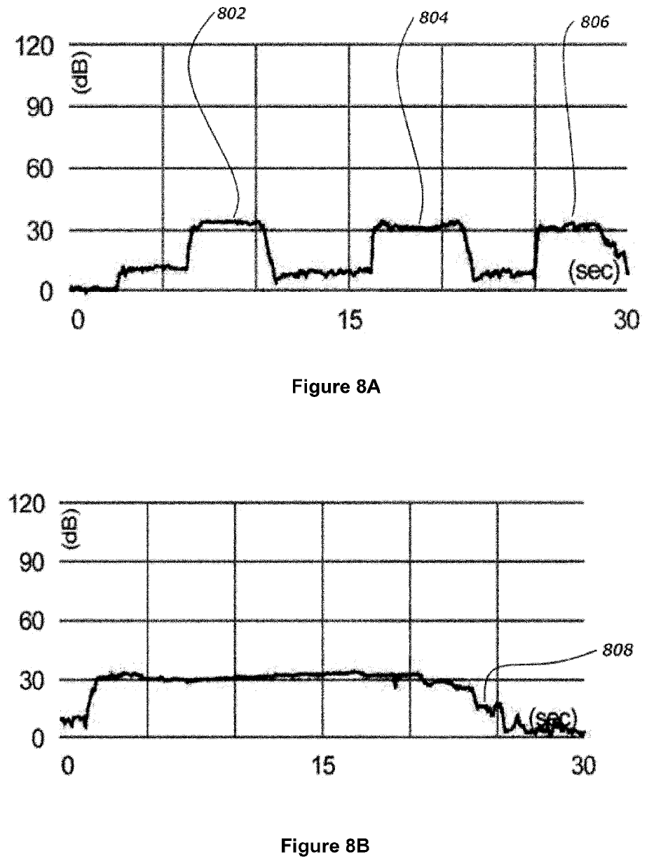

FIGS. 7a to 7c show three exemplary plots of Force applied by a user during an exercise and the corresponding reflected acoustic signal that is measured. The plots indicated in FIGS. 7a-7c show plots of the raw signal data prior to any processing. The raw signal may be presented to the user via the mobile device 200 in instances if the user selects such an option via the mobile device 200. The mobile device 200 may be configured to extract raw signals from the wearable device 100 and display them. Preferably the mobile device 200 provides feedback as shown in FIGS. 8a-8b or FIGS. 9-11.

The three FIGS. 7a to 7c illustrate how three different muscular parameters are determined by processing the reflected acoustic signal.

Referring to FIG. 7a the plot 702 represents the force applied during bicep curls. Each peak 710, 712, 714 represent the force applied by the bicep muscle during repetition of a bicep curl. The plot 704 represents the reflected acoustic signal. Muscular activation can be determined based on each peak being detected. The circles 720, 722, 724 represent muscle activation during each repetition of a bicep curl.

Referring to FIG. 7b muscle fatigue can be determined based on a trend of the peaks of the reflected acoustic signal. As seen in FIG. 7b the trend line 730 represents a decaying trend. A decaying trend represents muscular fatigue. If the amplitude of each successive peak of the reflected acoustic signal is reducing is indicative of muscular fatigue.

Referring to FIG. 7c muscular density can be determined based on the width of each peak i.e. the area under each peak. The squares 740, 742, 744 represent the area under the peak that is used to determine muscle density. In one example muscle density is determined based on the area of each square or based on the RMS or dB values of the signal. In another example the muscular density may be determined based on the value of the amplitude of the reflected acoustic signal e.g. the absolute value of the amplitude.

The muscular parameters are preferably determined by the wearable device 100. However the muscular parameters may be determined by the mobile device 200 and in some instances by the server 310. Preferably the muscular parameters are determined in substantially real time to allow substantially real time feedback to the user. Hence the muscular parameters are preferably determined by the wearable device 100 or the mobile device 200. The muscular parameter information can be used to provide feedback regarding exercise to a user.

FIGS. 8a and 8b show exemplary plots that are generated during exercise. FIGS. 8a, 8b show examples of the dB plot generated by server 310 when in use. Referring to FIG. 8a, there are three peaks 802, 804 and 806 that are illustrated. The peaks correspond to the increase in amplitude of the reflected acoustic signal due to contraction of the muscle. The amplitude of the peak corresponds to or is at least indicative of the contraction force applied. The amplitude of each peak of the absolute value of each peak may be related to the density of the muscle. The density of the muscle may be derived from the amplitude of the dB values. The wearable device 100 controller 106 is configured to process the reflected acoustic signals by filtering the signals using RMS to reduce noise in the signal. The signals are then converted into dB values using a suitable mathematical transform or function that is stored as part of the software application or as part of computer readable and executable instructions. The dB representation of sound provides a time domain representation.

The three peaks 802-806 shown in FIG. 8a, correspond to three bicep curls performed by the user. This information can be used to determine if the user is applying the correct amount of force i.e. lifting an appropriate weight that will align with the amount of work the user wants to perform. FIG. 8b shows an exemplary plot of a single contraction until failure. As can be seen in FIG. 8b, there is a failure point 808. The amplitude of the signal decays past failure point 808. The failure point represents failure of the muscle due to work i.e. contraction. These plots may be visually presented to the user on the mobile device 200, or alternatively feedback can be generated based on the plots of the reflected acoustic signal. Further the reflected acoustic signal is used to determine a muscular parameter e.g. muscle density, muscle fatigue, muscle activation or muscle engagement.

The system 10 provides feedback to users during training. The feedback may be live feedback during an exercise routine or may be provided post routine. The data during an exercise routine may be stored in a memory unit associated with the server 310 or alternatively may be locally stored in a memory unit of the mobile device 200. This data can allow a user to push themselves further during an exercise routine or allow a trainer to assess the needs of their client/athlete and either provide feedback live during an exercise routine or post exercise routine. The real time determination of muscular parameters by the wearable device 100 can allow for real time feedback during a workout or exercise routine for an athlete, thereby allowing the athlete or coach to assess real time performance but also allow them to make decisions regarding if the athlete needs to work harder, work less, change the workout or have a rest.

The system 10 using the device 100 to determine muscular parameters is suited for a common gym goer to provide feedback regarding their routine or alternatively for high level athletes to track their performance and customize exercise routines. The system 10 also allows for customized exercise routines to be selected by the user. For example the user can select training for reps or can select training for failure. The system 10 tracking the muscular parameters can provide feedback regarding the performance of a selected routine either live during the workout or post workout.

### Displayed Information Examples

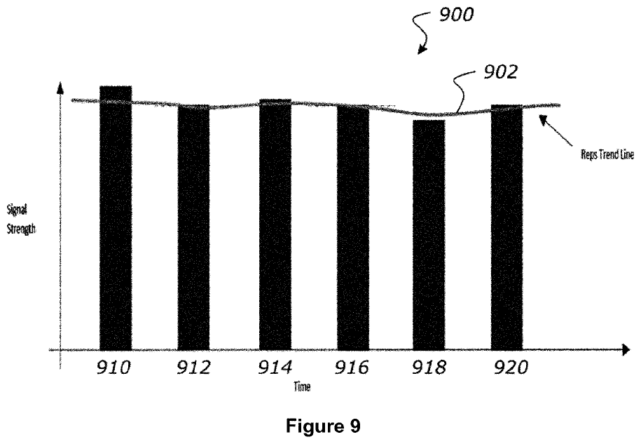

FIG. 9 shows an exemplary plot 900 that is generated during multiple repetitions. The user has selected an exercise routine to train for 6 repetitions and short of failure i.e. no failure should occur. The reflected acoustic signal at each rep is plotted on the plot 900. A trend line 902 can be plotted across the various peaks. Each peak 910-920 corresponds to a repetition of a bicep curl. The trend line 902 can be used to inform a user regarding their performance. In this example the user can be instructed to put more effort in or increase their weight. The plot 900 displays the consistency of each repetition. This allows a user or trainer or coach to assess either the user's technique or weight used and whether that is adequate.

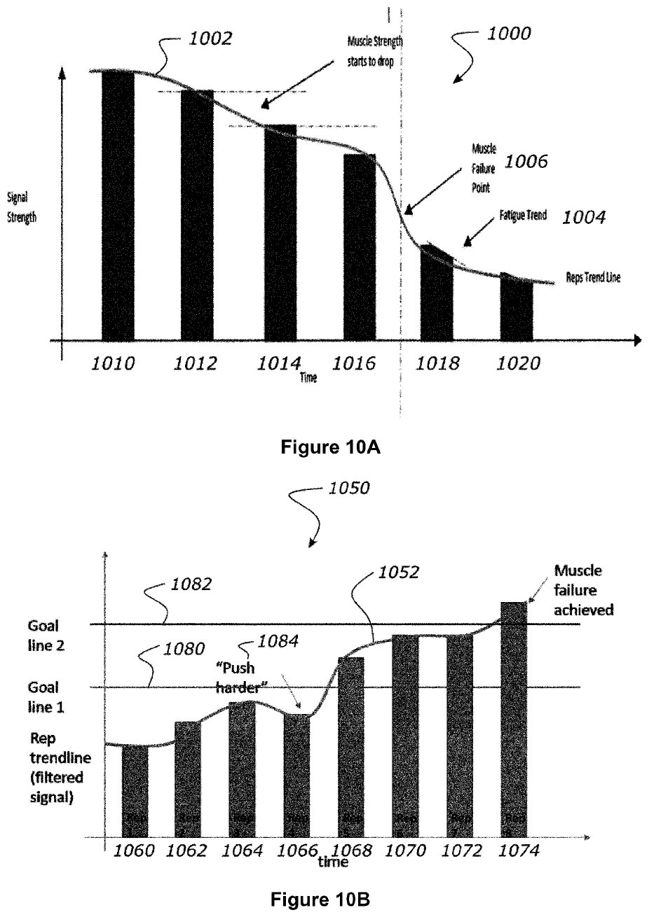

FIG. 10a shows an exemplary plot 1000 that is generated during multiple repetitions of bicep curls. The selected workout is to work out until muscular failure in the example of FIG. 10a. As can be seen in FIG. 10a, there are six peaks 1010-1020, each peak corresponding to a contraction of the muscle i.e. each peak corresponding to a bicep curl being performed. The trend line 1002 begins to drop off from repetition 1012 and continues to drop indicating the user is struggling and likely reaching a failure point 1006. The muscular strengths begins to drop between rep 1012 and 1014 since the user is exerting (i.e. contracting) as much as possible. The selected workout, as per plot 1000, is a failure type workout. The muscular strength is indicated by the amplitude of the reflected acoustic signal. Muscular strength may be indicated by muscular density that is determined based on the amplitude (or area under the curve) of the reflected acoustic signal.

Referring to FIG. 10a, at repetition 5 (1018) the amplitude is below a failure threshold and hence indicates muscular failure. The fatigue trend 1004 is illustrated which is a decay in the amplitude of the reflected acoustic signal. Muscle failure is characterized as being exhaustion of all the neurotransmitters within the muscle. Once muscle failure has been achieved, the muscle will not contract more than a particular amount e.g. 60% of the one rep max. This can be seen in FIG. 10a, where reps 1018 and 1020 amplitude does not increase i.e. the density does not increase since the muscle does not contract any further. The mobile device 200 may illustrated or present this graph. The graph may be presented in real time as each repetition is performed, and messages such as “muscle failure point” or “muscle strength dropping” may presented to the user visually or audibly or both.

FIG. 10b shows an exemplary plot 1050 that is generated during multiple repetitions of bicep curls. The selected workout is one to achieve muscular failure. As shown in FIG. 10b the trend line 1052 is plotted across the various repetition peaks 1060-1074. A first goal line 1080 can be indicated. The goal line may be the line that needs to be achieved by the user. The goal line 1080 is indicative of the effort that needs to be achieved by the user in order to achieve a predefined rate of effort for each repetition. Each peak represents a muscular contraction and an increase in density which manifests in an increase in the amplitude of the reflected acoustic signal. A second goal line 1082 is indicated that corresponds to a threshold to achieve muscular failure. Once the trend line crosses the second goal line 1082 the device 100 or mobile device 200 may provide an audible, visual or haptic feedback to the user that muscular failure has achieved. The device 100 tracking the density of the muscle i.e. each peak can provide feedback to the user e.g. “push harder” 1084 i.e. increase effort in order to achieve the specified goal. Push hard may mean increasing the effort or may mean increasing the weight being lifted to achieve the desired effort goal. In this example effort may relate to the muscular density for each repetition. The term effort may also relate to another muscular parameter that is determined by the wearable device 100.

Each goal line 1080, 1802 may relate to a separate threshold. Each threshold can relate to a specific fitness goal a user is aiming for. For example the first goal line 1080 may correspond to 80% effort of the one repetition maximum for a user. For example goal line one may relate to a muscle density corresponding to 80% of the one rep max of the user. The second goal line 1082 may relate to 90% or 100% of the one rep max thereby indicating the effort (i.e. muscle density) required to achieve muscular failure. In one example 80% can be performed to achieve muscular hypertrophy. For endurance training the goal line may be set at 30-40% of the one rep max. For powerlifting or failure the goal may be set at 95%. The goal line indicates a threshold for exercise. The user can predefine a threshold based on the results the user wishes to achieve. The wearable device 100 and system 10 provides real time feedback to a user based on the determined muscular parameters.

The plot 1000 shown in FIG. 10a is an example of muscular failure being achieved and detected during a workout. The muscular failure was not a goal of the workout shown in plot 1000. The plot 1050 shows an example of different thresholds being set by the user, and the user actively trying to achieve muscle failure, or at least achieve a threshold that will lead to muscle failure. If plot 1050 were to be extended out in time, it may begin to look like plot 1000 as muscular failure is achieved. The plots 1000 and 1050 may be presented visually on the mobile device along with audible feedback too.

### Exemplary Use

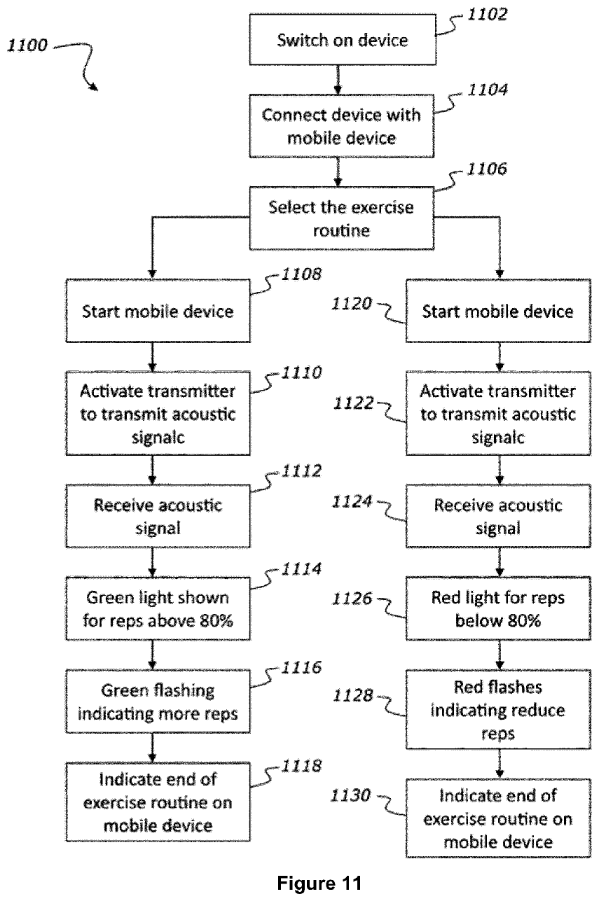

FIG. 11 shows an exemplary method of assisting a user during exercise 1100. The method 1100 comprises using the system 10 and its components. Step 1102 comprises switching on the wearable device 100. Step 1104 comprises pairing the wearable device 100 with the mobile device 200, which is a smartphone in this case. Step 1106 comprises selecting an exercise routine. In this example the user can select one of two exercise routines: 1. Training short of muscle failure e.g. for reps. 2. Training for muscle failure.

If the user selects routine 1 (training short of muscle failure), the method moves to step 1108. Step 1108 comprises starting mobile device 200. The mobile device 200 acts as a user interface for the user with the system 10. Step 1110 comprises activating the transmitter to transmit an acoustic signal to the user's muscle. In particular the acoustic signal is a vibroacoustic signal. Step 1112 comprises receiving the reflected acoustic signal during the exercise routine. Step 1114 comprises displaying a green light via one of the indicators 112, 114 if the amplitude of the reflected acoustic signal is above a threshold. The threshold may be any suitable arbitrary threshold selected by the user or by a trainer of the user. In this example the threshold is 80% of the one rep. maximum amplitude. The threshold may be calculated during a calibration process e.g. during step 1104 where the wearable device 100 and the mobile device 200 are paired together.

The threshold could be linked to the work out and could be based on the work out level or desired results of the user. For example if a user wants muscular hypertrophy, the user can set a corresponding threshold. Alternatively if the user wants endurance type workout the user can set a threshold that corresponds to endurance. A further example of a threshold may be the number of reps that are performed. A user can set multiple thresholds for a particular workout. For example the user may have to perform reps or perform reps where the amplitude exceeds a second threshold. The thresholds may be illustrated on the user interface such as that shown in FIG. 10b. A threshold is defined during a calibration process.

The green light flashes for each repetition performed by the user, if the reflected acoustic signal is greater than 80% of the maximum amplitude. Step 1116 comprises the indicator e.g. LED 112, 114 flashing green for each repetition performed where the amplitude of the reflected acoustic signal is greater than the threshold. Step 1118 indicates an end of the exercise routine. The end of the exercise routine may be shown on the mobile device 200 or by the indicators e.g. by showing a specified lighting sequence corresponding to the end of a routine. Alternatively if the indicators may provide an audible or haptic indication if the indicators are audible indicators or haptic indicators respectively. If the amplitude is less than a threshold e.g. 80% of the one rep max, there may be an appropriate indication instructing the user to work harder e.g. a flashing red light. If the processor (e.g. server 310) detects that the user muscle is fatiguing based on the muscle fatigue parameters detected i.e. the trend line, the mobile device 200 or the indicator may direct the user to stop during this workout. Muscle fatigue may be determined if the reduction in the amplitude of two or more acoustic signals corresponding to repetitions exceeds a threshold i.e. the amplitude is dropping to quickly an indicate muscle failure.

Still with reference to FIG. 11, if the user selects an exercise routine to train to failure at step 1106, the method moves to step 1120. At step 1120 the mobile device is activated. Step 1122 comprises activating the transmitter 102 and transmitting an acoustic signal, e.g. a vibroacoustic signal to the muscle of the user. Step 1124 comprises detecting the reflected acoustic signal. Step 1126 comprises indicating that a user is not producing enough force i.e. the muscle is failing. In this example if the amplitude is less than 80% of the one rep maximum indicates failure may be approaching. The processor e.g. server 310 may compute a trend line between consecutive repetitions to observe any trends. Step 1128 comprises flashing a red light indicating reduction in repetitions in order to prevent injury. This may also be an indication that failure has occurred. Step 1130 comprises the mobile device 200 and/or the wearable device 100 indicating an end of the selected exercise routine.

Prior to use of the system 10 and device 100 in exercise, a user must undergo a calibration process. The calibration process can determine various baseline parameters for a user. For example a one rep max can be determined as part of the calibration process. Various other baseline values may be determined e.g. resting heart rate, fat %, muscle density for a one rep max etc. The baseline parameters can be used to set various user defined thresholds.

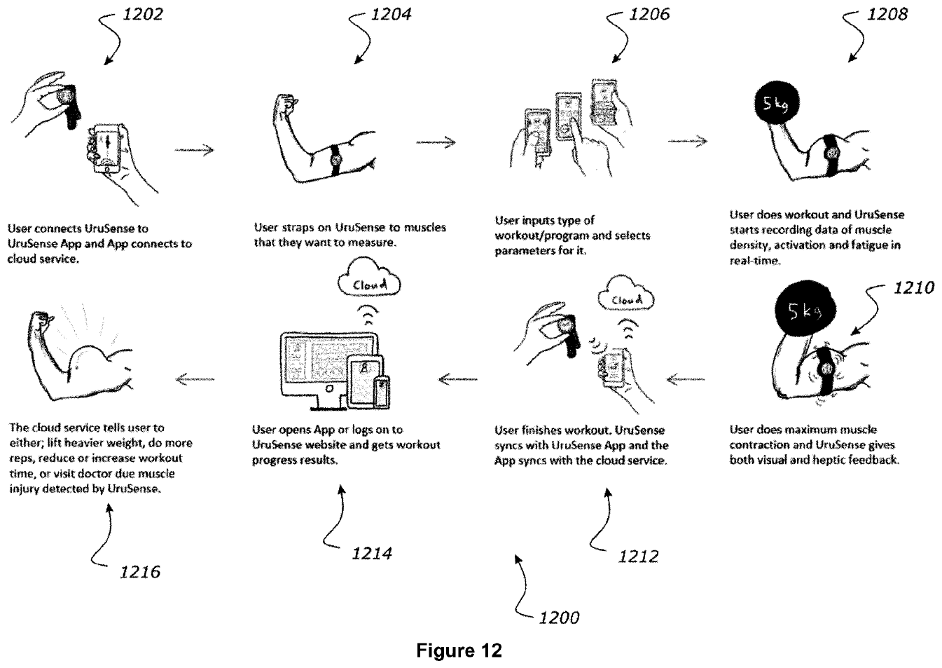

FIG. 12 shows an example method of using an exercise system 10 including a wearable device 100 and a mobile device 200, during exercising. FIG. 12 shows an example use case of the system 10 and the wearable device 100. The method of exercising 1200 as shown in FIG. 12 is executed by a user utilizing the system 10 and its components.

The method of exercising 1200 commences at step 1202. Step 1202 comprises pairing the wearable device 100 with the mobile device 200. The mobile device 200 preferably comprises an application (i.e. app) that is stored on the mobile device 200. The app defines computer executable instructions that control the operation of the mobile device 200. Step 1202 also comprises pairing or linking the mobile device with the server 310, via the app and wireless communication between the server 310 and the mobile device 200. Step 1204 comprises the user wearing the wearable device 100. The wearable device 100 is attached to the user's bicep in the illustrated example using the mounting assembly i.e. a strap 152. The wearable device 100 is positioned for determining a muscular parameter.

Step 1206 comprises the user selecting an exercise routine on the mobile device 200. The mobile device 200 preferably presents one or more exercise routines that are selectable via the user interface of the mobile device e.g. the touchscreen of the illustrated mobile device. The mobile device 200 acts as an interface between the user and the server 310 (i.e. the processing and storage system 300). The mobile device 200 is configured to receive a selection of one or more exercise routines to be performed by the user at step 1206.

Step 1208 comprises the user performing an exercise routine i.e. workout, as selected. The wearable device 100 begins to record the reflected acoustic signals and process them. Once processed the reflected acoustic signals may be transmitted to the mobile device 200.

Step 1210 comprises the user performing a muscle contractions as part of the exercise routine. The wearable device 100 is configured to determine one or more muscular parameters as the user performs the exercise routine. The muscular parameters are determined based on the reflected acoustic signal. The wearable device 100 or the mobile device 200 may provide real time feedback to the user as the user performs the workout.

The feedback provided to the user is preferably regarding one or more aspects of the selected exercise routine based on the determined muscular parameters. The aspect of the one or more exercise routines may be one or more of amount of weight to lift, number of repetitions, a change in the exercise routine duration, or stop exercising due to an injury. For example the wearable device may provide visual or haptic feedback to the user, in real time, as the user is performing an exercise routine. The feedback is preferably provided via the indicators 112, 114. The mobile device 200 may also be configured to provide feedback to the user regarding the user's performance of a selected exercise routine. The feedback may be provided by the wearable device 100 or mobile device 200 or both.

Step 1212 comprises transmitting the information regarding the exercise routine. The mobile device 200 may store measurements throughout the exercise routine. These measurements may be locally stored on the mobile device and then uploaded to server 310. Step 1212 comprises synchronizing i.e. pairing at least the mobile device 200 with the server 310. The app being executed on the mobile device 200 is used to synchronize with the server 310.

Step 1214 comprises providing a progress report regarding the progress of the user while performing the selected exercise routine. The information may be a log or a progress report or a summary or any other suitable form. The reports may be generated by the server 310 based on aggregated data and provided to the user. The reports may include daily peak values, maximums achieved, an average muscular density (i.e. effort) during works, a change in density which may represent a change in the muscle. The reports may also include progress reports e.g. a histogram showing data related to one or more workouts performed over a time period e.g. a week or month. Various muscular parameters that were determined during various workouts over a time period may also be generated and presented as part of a report.

At step 1216 the server 310 is configured to generate one or more recommendations regarding how the user can improve their performance in an exercise routine. This recommendation is based on the determined muscular parameter. The recommendation may also be based on how the muscular parameter has compared to a desired change during an exercise routine. The desired change is preferably pre-defined by a user. The recommendations may be for example, lift heavier, do more repetitions, reduce or increase duration of workout. The system can also determine if an injury has occurred based on the determined muscular parameter. The user can be alerted regarding an injury or if the user is likely to injure themselves. Injury detection and feedback can also be provided in real time. This is advantageous because the system 10 provides the user feedback regarding performance during an exercise routine, identify if an injury has occurred and/or identify how the user can improve their performance. This is particularly advantageous for trainers and coaches as it allows a real time manner of detecting performance during exercise.

The system 10 and its components have wide ranging applications and advantages, as outline below. The wearable device is advantageous because it allows for real time determination and feedback regarding an exercise. The wearable device provides a substantially non invasive and safe way of determining muscular parameters without the need for complex and large scale devices. The wearable device uses acoustic signals, in particular vibroacoustic frequency range signals which is safe protocol and does not damage the muscles. Further the use of vibroacoustic range signals substantially matches or is close to the natural resonant frequency of muscle fibers, hence improving reflection of the acoustic signal. This allows for a substantially accurate way of determining muscular parameters. The wearable device 100 is also advantageous because it provides a non invasive manner of determining a number of different physiological parameters, in particular various muscular parameters based on the reflected acoustic signals.

The wearable device 100 is advantageous because it is small, light and portable compared to known systems such as ultrasound or ECG or EMG. These known systems are not portable and require bulky, expensive and complex devices. Further the use of ultrasound for longer periods of time may cause damage to parts of the muscle, and ultrasound also may lead to a noisy signal being received as the reflected acoustic signal due to scattering of the ultrasound signals. The system and wearable device allow for real time feedback regarding the performance of a user and allows the user to customize their various workouts. The use of the wearable device that is configured to provide an indication regarding the performance of a user based on the determined muscular parameters provides an objective measure of effort put in by a user during exercise. For example determining a muscular parameter e.g. density per repetition provides a fairly accurate way to track the user's effort on each repetition and the visual/audible feedback from the wearable device 100 and/or mobile device 200 can reduce the chances of “cheating” on reps. The feedback provides a trainer or user feedback if the user is “cheating” on lifts which can help to maximize effort and effects of exercise on a user. The results a user wants to achieve are customizable by selecting the specific workout or result a user wants to achieve e.g. a percentage of their one rep max.

The system 10 is also advantageous because it stores muscular parameters (and other physiological parameters) determined based on the reflected acoustic signals (i.e. or an electronic signal indicative of the reflected acoustic signals). The stored muscular parameters allows a user to track changes within their own musculature based on changes in the recorded muscular parameters over a specified time period. The system 10 is advantageous because the time series changes to muscular parameters can be illustrated visually on at least the mobile device 200 or another user computing device e.g. a laptop or desktop. The user can login to access user related information using user credentials that are associated with the user, e.g. a username and password. The server 310 is configured for communication with a mobile device and/or a computing device. The user can access their own information to track long term changes to a muscular parameter. Long term changes to a muscular parameter can be indicative of changes in a user's physique. For example increase in base line muscular density may relate to an increase in muscle mass for a muscle group. The user may also access changes in muscular parameters for a plurality of muscle groups if a plurality of wearable devices are used, to indicate a change in each of the muscle groups. This information can help a user or train assess which areas need further work, assess how quickly particular muscle groups of a user are developing and help in customizing workouts based on a user or trainers goal.

### Exemplary Applications

The system 10 can be used for a number of different uses. One exemplary use is for the regular person who goes to the gym. The exercise system 10 allows a person to track their own progress based on the muscular parameters that are determined. The user can be provided with information about muscular change e.g. muscular hypertrophy. Further the system 10 provides real time feedback to the user while performing an exercise routine. The system 10 also allows a user to improve their performance based on the determined muscular parameters and the feedback that is provided.

Another industry that can use the system 10 is for sports training, especially in training high level athletes. Athletes of all types who engage in resistance training can benefit from the feedback provided. Determining the muscular parameters allows the athletes or their coaches to determine the type of effort and if there is adequate effort being applied by the athlete. The system 10 allows for a more accurate determination of performance during an exercise routine and the effect that the exercise is having on their muscles. The system 10 can also help in reducing overtraining and can help in reducing stress injuries to muscles since the system 10 measures muscular parameters and can detect if an injury has occurred. The system 10 also provides a portable, easy to transport system that can be used in any environment.

Muscle degradation assessment (sarcopenia) is common as it naturally happens from around 30 years of age and older. The wearable device 100 can assess the change in muscle density and activation of muscles between two different time points. This allows a clinician or doctor to assess the rate of degradation as well as assess if any therapeutic exercises are providing the desired results.

Musculo-skeletal rehab is a further application. The system 10, in particular the wearable device 100 can be used to assess the muscle density or other muscular parameters of an injured limb. The performance of an injured limb can be compared with that of a healthy limb providing a clinician or physiotherapist an accurate way to track if a user (i.e. patient in this case) is improving. In the case of a bilateral injury the performance of the injured limbs can be compared with a standard health limb value based on age and sex. The system 10 allows for an objective measure of muscular parameters and an objective measure of the rehabilitation process.

Work safety can be another potential application in order to assess if any workers are suffering injuries due to long term exposure to particular types of work e.g. heavy lifting. The system 10 allow for objective measurement of muscular performance and/or degradation. The system 10 can also be used to provide feedback to workers to prevent injury or detect when an injury occurred. These reports can be used to reduce work place injuries or at least identify what was a potential cause of this injury.

### Alternative Configurations

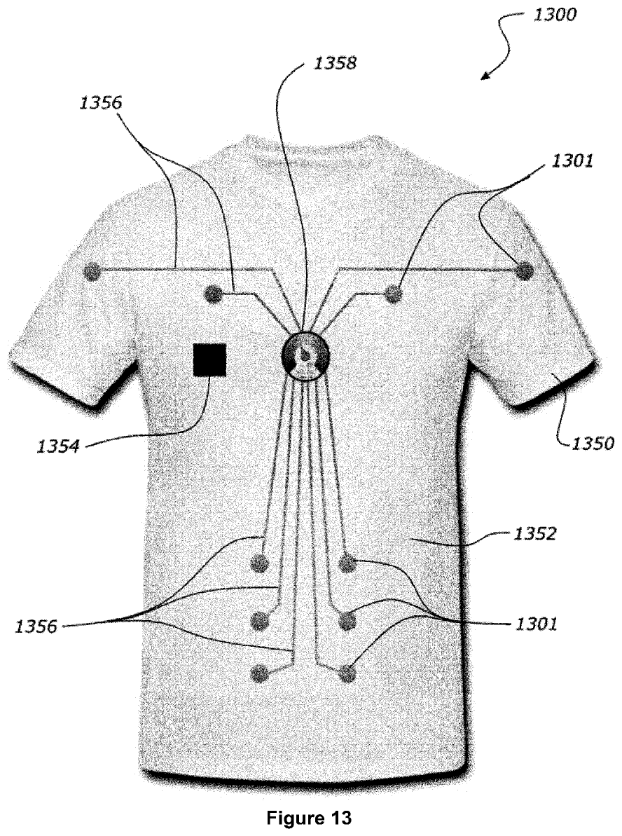

FIG. 13 shows an exemplary configuration of the wearable device 1300. The wearable device 1300 is an alternative embodiment to wearable device 100. The wearable device 1300 is used as part of an exercise system to assist a user performing exercises. The wearable device 1300 can also be used to determine a physiological parameter e.g. one or more muscular parameters. Referring to FIG. 13, the wearable device 1300 comprises one or more transducers 1301 disposed on a mounting assembly 1350. The one or more transducers 1301 comprise at least a transmitter and a receiver. The transmitter is configured to transmit acoustic signals in the form of vibrations. The receiver is configured to receive reflected acoustic signals. The acoustic signal frequency is in the vibroacoustic signal range as described earlier. The transducers 1301 may include a similar structure as the transducer 101 as described earlier.

The mounting assembly 1350 comprises a garment 1352, as shown in FIG. 13. The one or more transducers 1301 are disposed on or within the garment 1352. The one or more transducers 1301 may be adhered to the garment or sewn in the garment or may be located within pockets created in the garment. The one or transducers 1301 are positioned on or within the garment such that acoustic signals can be transmitted into a user's body e.g. into different muscle groups and reflected acoustic signals can be received by the transducer (i.e. by the receiver of the transducer). The garment 1352 may include cut outs within it to receive the one or more transducers 1301. Alternatively a layer of the garment 1352 may be located between a portion of the transducer and the skin of the user. For example a layer of the garment 1352 may be located between at least the transmitter and the skin. The garment 1352 may function as the contact layer 120 as described earlier.

The garment 1352 may be a shirt, a singlet, a t shirt or shorts or briefs. The garment can be worn by the user in use such that the transducer is brought adjacent a muscle or muscle group. As shown in FIG. 13, the garment 1352 is a t shirt. The garment 1352 preferably is a tight fitting garment i.e. a substantially figure hugging garment in order to bring the one or more transducers 1301 in contact with or as close to the skin as possible to improve coupling between the transducers 1301 and the skin of the user. In one example the garment 1352 may be a compression type garment. The transducers 1301 may be positioned at locations on the garment such that in use, the transducers 1301 align with or adjacent a major muscle or major muscle group e.g. biceps or triceps or quadriceps etc. The garment 1352 may be made from any suitable material such as cotton or nylon or spandex or a knitted fabric. The garment 1352 may also include wicking features to wick moisture or other cooling features to try and maintain a cooler body temperature for a user.

As shown in FIG. 13 the garment is a t-shirt. The garment includes a plurality of transducers 1301 (each being denoted by a circle). The garment 1352 also includes an electronics module 1354 that is disposed on or within the garment 1352. The electronics module houses the electronics including at least a controller, one or more indicators and a communications unit, wherein the controller includes processor and memory unit. Alternatively each transducer 1301 may comprise its own electronics module associated with each transducer to determine a muscular parameter for the particular muscle the transducer is associated with.

The garment 1352 also comprises a plurality of electronic traces or wires 1356. The wires 1356 may connect each transducer to an indicator 1358 located on the garment. The wires may also connect each transducer 1301 to the electronics module 1354. The wires may be woven into the garment or may be formed of flexible PCB material. The electronics module 1354 may include similar electronics as the transducer 100 as described with reference to FIG. 2.

Referring again to FIG. 13, the garment may also include an additional indicator 1358 located substantially in the centre the garment. As shown in the exemplary configuration of FIG. 13, the indicator is positioned in the chest of the user. The indicator 1358 may be a visual or audio or haptic indicator or a combination thereof. The indicator 1358 is controlled by the controller can be used to provide information to the user related to an exercise being performed based on the determined muscular parameter. The device 1300 is configured to process the reflected acoustic signals and determined a physiological parameter such as one or more muscular parameters. This information can be communicated to the mobile device 200 or the processing and storage system 300 via a communication network. The wearable device 1300 functions substantially in the same manner as wearable device 100 as described earlier.

In some alternate configurations the wearable device 100 or mobile device 200 may be configured to process the reflected acoustic signals by converting the signals to frequency domain e.g. by applying fast Fourier transforms or other frequency domain transforms. The frequency domain plot may be used to distinguish various types of body components e.g. the frequency domain plot or information can be used to distinguish between fat, muscle and skin. Each component of the human body has a different frequency response to an applied acoustic signal. Therefore applying a frequency domain transform allows determination of the frequency response to the applied acoustic signals. The frequency response information can be used to determine or distinguish various components of a limb to which the device 100 is applied e.g. skin, fat and muscle. The device 100 or mobile device 200 may be configured to determine the amount of each component e.g. amount of fat, amount of muscle etc. These amount measurements may be aggregated and stored at the server 310 to allow users to view changes to fat or muscle over a specified time period. For example a frequency domain transform may be used as part or a step in method 600.

In some alternative embodiments the server 310 may be configured to receive filtered reflected acoustic signals (i.e. electrical signals indicative of the reflected acoustic signals). The server 310 may receive the reflected acoustic signals from the wearable device 100 directly or via the mobile device 200. The server 310 may be configured to process the reflected acoustic signals to determine one or more physiological parameters, e.g. one or more muscular parameters. The server 310 may be further configured to transmit the determined one or more physiological parameters to the mobile device 200 for presentation to the user and/or to the wearable device to activate one or more indicators on the wearable device 100. Currently as described earlier the processing of the reflected acoustic signals is performed by the wearable device 100 or by the mobile device. In this alternative configuration the processing of reflected acoustic signals is performed by the server 310 or processing and storage system 300 (as described earlier). The server 310 or an element of system 300 may constantly determine a physiological parameter e.g. a muscular parameter and transmit this information to the mobile device 200 and/or the wearable device. The determination and transmission of the physiological parameters maybe performed in real time or at a time interval substantially equivalent to real time e.g. less than every 5 seconds. The processing and storage system 300 may include one or more servers configured to perform the processing. The servers may be remote servers or geographically separated or may be implemented within partitioned processors or computing devices. The one or more servers may be implemented as cloud servers across a cloud service provider. The processing and storage system 300 further includes one or more memory units for storing information such as user credentials and the determined physiological parameters e.g. muscular parameters.

Where in the foregoing description reference has been made to elements or integers having known equivalents, then such equivalents are included as if they were individually set forth.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting. Where specific integers are mentioned herein which have known equivalents in the art to which this invention relates, such known equivalents are deemed to be incorporated herein as if individually set forth.

Although the invention has been described by way of example and with reference to particular embodiments, it is to be understood that modifications and/or improvements may be made without departing from the scope or spirit of the invention.

1. A device comprising:
a transducer;
a mounting assembly coupled to the transducer and configured to mount the transducer adjacent to or in contact with a portion of a user's body;
the transducer comprises:
a transmitter configured to generate and transmit an acoustic signal to a portion of the user's body; and
a receiver spaced apart from the transmitter by between 1 mm to 100 mm configured to receive a reflected acoustic signal, and
a processor in electronic communication with the transducer, the processor configured to determine a physiological parameter based on the reflected acoustic signal.

2. The device of claim 1 wherein the acoustic signal transmitted by the transducer is a low frequency acoustic signal or a vibroacoustic signal.

3. The device of claim 1 wherein the acoustic signal is of a frequency less than or equal to 250 Hz.

4. The device of claim 1 wherein the acoustic signal is of a frequency greater than or equal to 50 Hz and less than or equal to 500 Hz.

5. (canceled)

6. The device of claim 1 wherein the transducer is a vibroacoustic transducer capable of generating and receiving a vibroacoustic signal.

7-10. (canceled)

11. The device of claim 1 further comprising a housing, the transmitter and the receiver being located within the housing and being spaced apart from each other, wherein the housing comprises an opening positioned within the housing and adjacent the receiver.

12. The device of claim 11 wherein the opening defines an air gap adjacent the receiver.

13. The device of claim 1 wherein the device comprises a coupling layer disposed between the transducer and a portion of the user's body adjacent the transducer.

14. The device of claim 13 wherein the coupling layer is formed from a resilient material.

15. The device of claim 13 wherein the coupling layer is formed from at least one or a combination of the group comprising silicone material, elastomer, elastomeric material, rubberised material, silicone rubber material and a thermoplastic material.

16. The device of claim 13 wherein the coupling layer is adhered or attached to a user proximal face of the housing.

17. (canceled)

18. The device of claim 13 wherein the coupling layer can be removably attached to a portion of the user's body or a face of the housing.

19. The device of claim 13 wherein the coupling comprises an aperture within it, the aperture being aligned with the opening in the housing when the coupling layer is adhered or attached to the housing.

20. The device of claim 13 wherein the at least one physiological parameter is determined based on a mathematical relationship between the physiological parameter and reflected acoustic signal.

21. The device of claim 13 wherein the at least one physiological parameter is a muscular parameter or a fat parameter or a bone density parameter.

22. The device of claim 20 wherein the muscular parameter is any one of:
muscle density, muscle fatigue, muscle activation or muscle engagement.

23-26. (canceled)

27. The device of claim 20 wherein the mathematical relationship is a proportional relationship between an amplitude of the reflected acoustic signal and a muscle density, wherein muscle density is proportional to the amplitude of the reflected acoustic signal.

28-31. (canceled)

32. A system for determining a physiological parameter of a user comprising:
the device according to claim 1; and
a remote processor in communication with the device, the processor receiving the reflected acoustic signal or a signal indicative of the reflected acoustic signal and the processor further configured to determine at least one physiological parameter based on a parameter of the reflected acoustic signal, wherein the processor is separate from the device;
a mobile device in wireless communication with the device, the mobile device providing feedback to the user based on one or more muscular parameters detected from the reflected acoustic signal by the processor, the mobile device comprising a user interface for communication with the user, the mobile device presenting one or more exercise routines to the user via the user interface, the mobile device receiving a selection of one or more exercise routines to be performed by the user and the mobile device providing feedback regarding an aspect of the one or more exercise routines based on the one or more determined muscular parameters.

33-54. (canceled)

55. A method for determining a physiological parameter comprising the steps of:
providing an acoustic signal to a muscle or muscle group;
receiving a reflected acoustic signal; and
processing the reflected acoustic signal to determine a physiological parameter based on the reflected acoustic signal.

56. The method of claim 55 wherein the reflected acoustic signal is reflected off at least the muscle or muscle group of a user.

57-85. (canceled)
